(19) United States
(12) Patent Application Publication
(10) Pub. No.: US 2026/0209339 A1
(43) Pub. Date: Jul. 23, 2026

(54) MULTIVALENT AND MULTISPECIFIC CYTOKINE ANTAGONISTS

(71) Applicant: Radiant Biotherapeutics Inc., Montreal (CA)

(72) Inventors: Joanne Hulme, Toronto (CA); Peter Bayliss, Toronto (CA)

(21) Appl. No.: 19/140,335

(22) PCT Filed: Dec. 18, 2023
(86) PCT No.: PCT/CA2023/051687

§ 371 Date: Jun. 17, 2025

Related U.S. Application Data
(60) Provisional application No. 63/433,476, filed on Dec. 18, 2022.

(51) Int. Cl. C07K 16/24; A61P 35/00; C07K 1/107

(52) U.S. Cl. CPC C07K 16/248; A61P 35/00; C07K 1/1075; C07K 16/244

(57) ABSTRACT

Fusion polypeptides comprising: (1) a cytokine-binding moiety and (2) a nanocage monomer or subunit thereof; self-assembled polypeptide complexes comprising such fusion polypeptides. Self-assembled polypeptide complexes comprising Fc fusion polypeptides and fusion polypeptides disclosed herein and/or tumor-binding fusion polypeptides, and related compositions and methods.

### CROSS-REFERENCE TO RELATED APPLICATIONS

This application claims the benefit of and priority to U.S. Provisional Patent Application No. 63/433,476 (filed on Dec. 18, 2022), the disclosure of which is incorporated by reference herein in its entirety for all purposes.

### SEQUENCE LISTING

The present specification makes reference to a Sequence listing which has been submitted electronically in XML format and is hereby incorporated by reference in its entirety. Said XML file, created on Dec. 13, 2023, is named RBT-052WO_SL.xml, and is 330 kb in size.

### BACKGROUND

Cytokines are small proteins which have diverse roles in controlling growth and activity of cells such as immune cells and tumor cells. Anti-cytokine therapy is an emerging treatment paradigm for a variety of human diseases and conditions, including cancer and autoimmune disease. One challenge faced by this treatment modality stems from the complex disease biology, which may involve multiple pathophysiological processes driven by distinct cytokines. Therefore, therapeutic agents which can only target an individual cytokine may lack efficacy.

There is a need for anti-cytokine therapeutics with improved efficacy.

### SUMMARY

The present invention addresses this need with the provision of self-assembled polypeptide complexes whose specificities and valences can be tuned to enhance efficacy. In certain embodiments, the provided complexes are multispecific, e.g., capable of binding (e.g., specifically binding) to more than one cytokine. Provided self-assembled polypeptide complexes generally display one or more types of cytokine-binding moieties and optionally display one or more tumor-binding moieties. Such self-assembled polypeptide complexes may be used to sequester cytokines, thereby interfering with cytokine signaling. Also disclosed are related fusion proteins, complexes, compositions, and methods.

In one aspect, provided are fusion polypeptides comprising: (1) a cytokine-binding moiety and (2) a nanocage monomer or subunit thereof.

In some embodiments, the cytokine-binding moiety comprises an antibody or antigen-binding fragment thereof.

In some embodiments, the antibody or antigen-binding fragment thereof comprises an immunoglobulin heavy chain variable region (V_{H}) and an immunoglobulin light chain variable region (V_{L} or V_{K}).

In some embodiments, the antibody or antigen-binding fragment thereof comprises a Fab fragment.

In some embodiments, the Fab fragment is a single-chain Fab fragment (scFab).

In some embodiments, the cytokine-binding moiety is capable of binding to interleukin-6 (IL-6) or interleukin-11 (IL-11).

In some embodiments, the cytokine-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of an IL-6 or IL-11 antibody.

In some embodiments, the CDRs of the V_{H} and the V_{L} or V_{K} have sequences which are identical to those of the heavy and light chain CDRs of an IL-6 or IL-11 antibody, except for one or two amino acid substitutions total across all six CDRs.

In some embodiments, the CDRs of the V_{H} and the V_{L} or V_{K} are identical to those of the heavy and light chain CDRs of an IL-6 or IL-11 antibody.

In some embodiments, the cytokine-binding moiety comprises an antibody fragment comprising heavy and light chain variable regions with sequences that have at least 85% identity to those of the heavy and light chain variable regions of an IL-6 or IL-11 antibody.

In some embodiments, the cytokine-binding moiety is capable of binding to an epitope of interleukin-6 (IL-6), which epitope is involved in binding of IL-6 to IL-6 receptor (IL-6Rα).

In some embodiments, the cytokine-binding moiety is capable of binding to an epitope of IL-6 that is involved in binding of IL-6 to glycoprotein 130 (gp130).

In some embodiments, the nanocage monomer is a ferritin monomer or a subunit thereof.

In some embodiments, the ferritin monomer is a human ferritin monomer.

In some embodiments, the ferritin monomer is a ferritin light chain.

In some embodiments, the cytokine-binding moiety is linked via an amino acid linker to the nanocage monomer or subunit thereof.

In some embodiments, the cytokine-binding moiety is linked via the N-terminus of the nanocage monomer or subunit thereof.

In one aspect, provided are a self-assembled polypeptide complexes comprising: (a) a plurality of first cytokine-binding fusion polypeptides, each first cytokine-binding fusion polypeptide being a fusion polypeptide as disclosed herein; and (b) a plurality of Fc fusion polypeptides, each Fc fusion polypeptide comprising (1) an Fc polypeptide and (2) a nanocage monomer or subunit thereof.

In some embodiments, the self-assembled polypeptide complex further comprises (c) a plurality of second cytokine-binding fusion polypeptides, each second cytokine-binding fusion polypeptide being a fusion polypeptide as disclosed herein, wherein the first and second cytokine-binding fusion polypeptides comprise first and second cytokine-binding moieties, respectively, which are capable of binding to different epitopes.

In some embodiments, the first and second cytokine-binding moieties are capable of binding to different cytokines.

In some embodiments, the first cytokine-binding moiety is capable of binding to IL-6 and the second cytokine-binding moiety is capable of binding to IL-11.

In some embodiments, the self-assembled polypeptide complex further comprises (d) a plurality of tumor-binding fusion polypeptides, each tumor-binding fusion polypeptide comprising (1) tumor-binding moiety and (2) a nanocage monomer or subunit thereof.

In some embodiments, within each Fc fusion polypeptide, the Fc polypeptide is linked via an amino acid linker to the nanocage monomer or subunit thereof.

In some embodiments, the Fc polypeptide is linked via the N-terminus of the nanocage monomer or subunit thereof.

In some embodiments, the Fc polypeptide is linked via the C-terminus of the nanocage monomer or subunit thereof.

In some embodiments, within each tumor-binding fusion polypeptide, the tumor-binding moiety is linked via an amino acid linker to the nanocage monomer or subunit thereof.

In some embodiments, the tumor-binding moiety is linked via the N-terminus of the nanocage monomer or subunit thereof.

In some embodiments, the tumor-binding moiety comprises an antibody or antigen-binding fragment thereof.

In some embodiments, the antibody or antigen-binding fragment thereof comprises an immunoglobulin heavy chain variable region (V_{H}) and an immunoglobulin light chain variable region (V_{L} or V_{K}).

In some embodiments, the antibody or antigen-binding fragment thereof comprises a Fab fragment. In some embodiments, the Fab fragment is a single-chain Fab fragment (scFab).

In some embodiments, the tumor-binding moiety is a PD-L1-binding moiety.

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a PD-L1 antibody.

In some embodiments, the CDRs of the V_{H} and the V_{L} or V_{K} have sequences which are identical to those of the heavy and light chain CDRs of a PD-L1 antibody, except for one or two amino acid substitutions total across all six CDRs.

In some embodiments, the CDRs of the V_{H} and the V_{L} or V_{K} are identical to those of the heavy and light chain CDRs of a PD-L1 antibody.

In some embodiments, the tumor-binding moiety comprises an antibody fragment comprising heavy and light chain variable regions with sequences that have at least 85% identity to those of the heavy and light chain variable regions of a PD-L1 antibody.

In some embodiments, the nanocage monomers within each Fc fusion polypeptide, each cytokine-binding fusion polypeptide, and/or each tumor-binding fusion polypeptide are each a ferritin monomer or a subunit thereof.

In some embodiments, the ferritin monomer is a human ferritin monomer.

In some embodiments, the ferritin monomer is a ferritin light chain.

In some embodiments, the self-assembled polypeptide complex does not comprise any ferritin heavy chains or subunits of ferritin heavy chains.

In some embodiments, the self-assembled polypeptide complex does not comprise any iron-binding moieties.

In one aspect, provided are a pharmaceutical compositions comprising a self-assembled polypeptide complex as disclosed herein and a pharmaceutically acceptable excipient.

In one aspect provided are uses of a self-assembled polypeptide complex or a pharmaceutical composition as disclosed herein to treat, ameliorate, or prevent a disease or condition in a subject.

In one aspect, provided are a methods of treating, ameliorating, or preventing a disease or condition, the method comprising administering to a subject a self-assembled polypeptide complex or a pharmaceutical composition as disclosed herein.

In some embodiments, the subject is a mammal, e.g., a human.

In some embodiments, the disease or condition is cancer, e.g., a solid cancer or a hematological cancer.

In some embodiments, the disease or condition is an autoimmune disease or condition.

### BRIEF DESCRIPTION OF THE DRAWINGS

FIG. 1A is a diagrammatic representation of human ferritin light chain (hFTL) and example N-half ferritin (N-hFTL) and C-half ferritin (C-hFTL) molecules.

FIGS. 1B and 1C are diagrammatic representations of sets of fusion polypeptides that together may form exemplary Multabodies of the disclosure.

FIGS. 2A-2F show representative Octet binding sensorgram plots from experiments to measure the interaction affinity and kinetics between IL-6 and one of siltuximab immunoglobulin (IgG) (FIG. 2A), clazakizumab IgG (FIG. 2B), olokizumab IgG (FIG. 2C), Siltuximab Multabody (MB) (FIG. 2D), Clazakizumab MB (FIG. 2E) or Olokizumab MB (FIG. 2F).

FIGS. 3A-3C show IL-6 binding curves from ELISA experiments for anti-IL-6 Multabodies and corresponding parental anti-IL-6 IgGs: siltuximab (FIG. 3A), clazakizumab (FIG. 3B), and olokizumab (FIG. 3C). Also shown are IL-11 and bovine serum albumin (BSA)-binding curves for the same anti-IL-6 Multabodies and corresponding parental anti-IL-6 IgG.

FIG. 4 shows a Western blot of extracts from HCT-15 tumor cells incubated with IL-6 alone or together with anti-IL-6 Multabodies. The blot was stained for STAT3 to detect of phosphorylation of STAT3 (pSTAT3), a marker of IL-6-induced activation of IL-6Rα signaling. Actin staining is also depicted as a loading control.

FIGS. 5A-5C show dose-response curves for an IL-6 signal transduction reporter assay conducted for cells incubated with anti-IL-6 Multabodies or parental anti-IL-6 IgGs: siltuximab (FIG. 5A), clazakizumab (FIG. 5B), and olokizumab (FIG. 5C).

FIGS. 6A and 6B show dose-response curves for a STAT3 activation assay (Meso Scale Discovery) performed on HCT-15 colon cancer cells (FIG. 6A) or THP-1 leukemia monocytic cells (FIG. 6B) incubated with anti-IL6 Multabodies (MB) or with corresponding parental anti-IL6 antibodies (IgG). The parental antibody was olokizumab.

FIGS. 7A and 7B show dose-response curves for a cell proliferation assay (CellTiter-Glo Assay) performed on IL-6-dependent cells (INA-6 cells) incubated with anti-IL6 Multabodies (MB) or with corresponding parental anti-IL6 antibodies (IgG). Parental antibodies were siltuximab (FIG. 7A) and olokizumab (FIG. 7B).

FIG. 8 shows the binding profile of anti-IL-11 MBs and IgGs to IL-11 or IL-6 antigen as determined by ELISA.

FIG. 9 shows a dose-response of for a STAT3 activation assay (Meso Scale Discovery) performed on Panc-1 pancreatic cancer cells incubated with anti-IL-11 Multabodies (MB) or with corresponding parental anti-IL-11 antibodies (IgG). The parental antibody was TPP-29536.

### DETAILED DESCRIPTION

Disclosed herein are fusion polypeptides each comprising (1) a nanocage monomer or subunit thereof and (2) an antibody fragment (such as a cytokine binder or a tumor binder) or an Fc polypeptide. The nanocage monomers or subunits thereof drive self-assembly of the fusion polypeptides into complexes which display one or more of the aforementioned antibody fragments and Fc polypeptides.

### Definitions

The terms “about” and “approximately,” when used herein in reference to a value, are used interchangeably and refer to a value that is similar to the referenced value. In general, those skilled in the art, familiar with the context, will appreciate the relevant degree of variation encompassed by “about” or “approximately” in that context. For example, in some embodiments, the terms “about” and “approximately” may encompass a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the terms “alter,” “altered,” “decrease,” “decreased,” “increase,” “increased,” or “reduction,” “reduced,” (e.g., in reference to certain outcomes or effects) have meanings relative to a reference level. In some embodiments, in the context of discussing mutations in an Fc chain or Fc polypeptide, the reference level is a level known or as determined with an IgG that does not contain the referenced mutation(s) in the Fc region.

As used herein, the term “antigen binding fragment” of an antibody, refers to one or more fragments of an antibody that retain the ability to specifically bind to an antigen. Examples of binding fragments encompassed within the term “antigen binding fragment” of an antibody include a Fab fragment, a F(ab′)2 fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

As used herein, the term “binding,” unless otherwise specified, refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell). As used herein, the phrases “non-binding” or “no binding,” or similar phrases, between two entities refers to 1) a lack of detectable binding or 2) binding below a set threshold that corresponds to no binding in an appropriate assay, e.g., an in vitro binding assay such as biolayer interferometry, surface plasmon resonance, a cell binding assay such as flow cytometry, or enzyme-linked immunosorbent assay (ELISA). For example, in some embodiments, in an in vitro biolayer interferometry assay, a maximal association binding response of less than 0.1 nm after 180 seconds to a biosensor loaded with 0.8 nm of target when the test article is present at a concentration of 20 nM is classified as “non-binding.”

The terms “ferritin” and “apoferritin” are used interchangeably herein and generally refer to a polypeptide (e.g., a ferritin chain) that is capable of assembling into a ferritin complex which typically comprises 24 protein subunits. In some embodiments, the ferritin is a human ferritin, e.g., a human ferritin light chain, e.g., a human ferritin light chain having at least 85% sequence identity to SEQ ID NO:1 or UniProt P02792. In some embodiments, the ferritin is a wild-type ferritin. For example, the ferritin may be a wild-type human ferritin.

The term “ferritin monomer,” is used herein to refer to a single chain of a ferritin that, in the presence of other ferritin chains, is capable of self-assembling into a polypeptide complex comprising a plurality of ferritin chains, e.g., 24 or more ferritin chains.

As used herein, the term “linker” is used to refer to an entity that connects two or more elements to form a multi-element agent. For example, those of ordinary skill in the art appreciate that a polypeptide (e.g., fusion polypeptide) whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, the linker is an “amino acid linker,” that is, it comprises amino acid residues, e.g., an amino acid linker may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acid residues. In some embodiments, a linker is characterized in that it tends not to adopt a rigid three-dimensional structure, but rather provides flexibility to the polypeptide.

The term “multispecific,” as used herein, refers to the characteristic of having at least two binding sites at which at least two different binding partners, e.g., an antigen or receptor (e.g., Fc receptor), can bind. For example, a polypeptide complex that comprises at least two Fab fragments, wherein each of the two Fab fragments is capable of binding (e.g., specifically binding) to a different antigen, is “multispecific.” As an additional example, a polypeptide complex that comprises an Fc fragment (which is capable of binding (e.g., specifically binding) to an Fc receptor) and a Fab fragment (which is capable of binding (e.g., specifically binding) to an antigen) is “multispecific.”

The term “multivalent,” as used herein, refers to the characteristic of having at least two binding sites at which a binding partner, e.g., an antigen or receptor (e.g., Fc receptor), can bind. The binding partners that can bind to at least two binding sites may be the same or different.

The term “nanocage monomer,” as used herein, refers to a single chain of a polypeptide that is capable of self-assembling with other nanocage monomers to form a self-assembled polypeptide complex comprising a plurality of nanocage monomers. In some embodiments, the nanocage monomer is selected from monomers of ferritin, apoferritin, encapsulin, sulfur oxygenase reductase (SOR), lumazine synthase, pyruvate dehydrogenase, carboxysome, vault proteins, GroEL, heat shock protein, E2P coat protein, MS2 coat protein, fragments thereof, and variants thereof.

The term “polypeptide,” as used herein, generally has its art-recognized meaning of a polymer of at least three amino acids, e.g., linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, glycosylation etc. In some embodiments, proteins may comprise natural amino acids, non-natural amino acids, synthetic amino acids, and combinations thereof.

As used herein, the term “specifically binds, “specifically binding,” “binds specifically,” or similar terms, means that a binding moiety (e.g., an antibody or an antigen-binding fragment thereof) forms a complex with an antigen that is relatively stable under physiologic conditions. Specific binding can be characterized by an equilibrium dissociation constant of 1×10⁻⁶ M or less, 1×10⁻⁷ M or less, 1×10⁻⁸ M or less, or 1×10⁻⁹ M or less (e.g., a smaller K_{D} denotes a tighter binding). Methods for determining whether two molecules specifically bind are well known in the art and include, for example, equilibrium dialysis, surface plasmon resonance, enzyme-linked immunosorbent assay, or biolayer interferometry measurements, etc. In some embodiments, “specifically binds” and similar terms refers to a characteristic of the binding moiety in that the binding moiety is capable of binding to a target antigen but does is not capable of binding to other antigens such as distantly related family members of the antigen.

The term “self-assembled,” when used in reference to a macromolecular complex (e.g., a polypeptide complex), refers to the spontaneous formation of that complex when sufficient constituents of the complex (e.g., fusion polypeptides) to be formed are present. In some embodiments, complexes self-assemble in physiological conditions, or in a buffer (e.g., a solution) that corresponds to physiological conditions.

As used herein, the term “subject” to an organism, typically a mammal (e.g., a human). In some embodiments, a subject is suffering from or susceptible to a relevant disease, disorder or condition. In some embodiments, a subject displays one or more symptoms or characteristics of a disease, disorder or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered.

As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### A. Fusion Polypeptides

In certain embodiments, fusion polypeptides compatible with compositions and methods disclosed herein generally comprise (1) a nanocage monomer (or subunit thereof) as described herein and (2) a cytokine-binding moiety, a tumor-binding moiety, or an Fc polypeptide, which may be linked via a linker, such as a linker described herein.

### 1. Nanocage Monomers and Subunits Thereof

In some embodiments, the nanocage monomer is a ferritin monomer.

The term “ferritin monomer,” is used herein to refer to a single chain of a ferritin that, in the presence of other ferritin chains, is capable of self-assembling into a polypeptide complex comprising a plurality of ferritin chains, e.g., 24 or more ferritin chains. In some embodiments, the ferritin monomer is a ferritin light chain. In some embodiments, the ferritin monomer does not include a ferritin heavy chain or other ferritin components capable of binding to iron or capable of ferroxidase activity.

In some embodiments, each fusion polypeptide within the self-assembled polypeptide complex comprises a ferritin light chain or a subunit of a ferritin light chain. In these embodiments, the self-assembled polypeptide complex does not comprise any ferritin heavy chains or subunits of ferritin heavy chains. In such embodiments, the self-assembled polypeptide complex does not comprise and is not associated with Fe³⁺, an iron ion normally carried by ferritin complexes which include the ferritin heavy chain.

In some embodiments, the ferritin monomer is a human ferritin chain, e.g., a human ferritin light chain, e.g., a human ferritin light chain having the sequence of at least residues 2-175 of SEQ ID NO:1.

In some embodiments, the ferritin monomer is a full-length human ferritin light chain. As used herein, a “full-length ferritin light chain” refers to a ferritin polypeptide that is capable of forming a four-helix bundle of two couples of anti-parallel α-helices connected by a loop and further comprising a C-terminal α-helix. In some embodiments, a full-length ferritin light chain comprises at least 85%, at least 90%, at least 95%, or 100% of the length of an amino acid sequence of SEQ ID NO: 1. In some embodiments, a full-length ferritin light chain has an amino acid sequence which has at least 85%, at least 90%, or at least 95% sequence identity to the SEQ ID NO: 1. In some embodiments, a full-length ferritin light chain has an amino acid sequence of SEQ ID NO: 1. In some embodiments, the full-length ferritin light chain is a single contiguous ferritin polypeptide.

A “subunit” of a ferritin monomer refers to a portion of a ferritin monomer that is capable of spontaneously associating with another, distinct subunit of a ferritin monomer, so that the subunits together form a ferritin monomer, which ferritin monomer, in turn, is capable of self-assembling with other ferritin monomers to form a polypeptide complex.

In some embodiments, the ferritin monomer subunit comprises approximately half of a ferritin monomer. As used herein, the term “N-half ferritin” refers to approximately half of a ferritin chain, which half comprises the N-terminus of the ferritin chain. As used herein, the term “C-half ferritin” refers to approximately half a ferritin chain, which half comprises the C-terminus of the ferritin chain. The exact point at which a ferritin chain may be divided to form the N-half ferritin and the C-half ferritin may vary depending on the embodiment. In the context of ferritin monomer subunits based on human ferritin light chain, for example, the halves may be divided at a point that corresponds to a position between about position 75 to about position 100 of SEQ ID NO:1 (or a substantial portion thereof). For example, in some embodiments, an N-half ferritin based on a human ferritin light chain has an amino acid sequence corresponding to residues 1-95 of SEQ ID NO: 1 (or a substantial portion thereof, e.g., residues 2-95 of SEQ ID NO: 1), and a C-half ferritin based on a human ferritin light chain has an amino acid sequence corresponding to residues 96-175 of SEQ ID NO: 1 (or a substantial portion thereof).

In some embodiments, the halves are divided at a point that corresponds to a position between about position 85 to about position 92 of SEQ ID NO: 1. For example, in some embodiments, an N-half ferritin based on a human ferritin light chain has an amino acid sequence corresponding to residues 1-90 of SEQ ID NO:1 (or a substantial portion thereof, e.g., residues 2-90 of SEQ ID NO: 1), and a C-half ferritin based on a human ferritin light chain has an amino acid sequence corresponding to residues 91-175 of SEQ ID NO:1 (or a substantial portion thereof.

### 2. Binding Moieties

Binding moieties (e.g., cytokine-binding moieties or tumor binding-moieties) typically comprise an antibody fragment.

In some embodiments, the antibody fragment is a Fab. In some embodiments, the antibody fragment is a single-chain Fab (scFab); for example, a fusion polypeptide comprising both the heavy and light chains of a Fab, optionally linked by a linker (e.g., amino acid linker as disclosed herein) is used.

In certain embodiments, the antibody fragment comprises a heavy chain variable region (e.g., a V_{H}). In certain embodiments, the antibody fragment comprises a heavy chain variable domain (e.g., V_{H}) and a light chain variable domain (e.g., a V_{L} or V_{K}). In certain embodiments, the antibody fragment comprises a Fab which comprises a heavy chain variable domain (e.g., V_{H}) and a light chain variable domain (e.g., a V_{L} or V_{K}).

In certain embodiments, the antibody fragment does not comprise any domains from the Fc region, e.g., does not comprise any constant heavy (CH2 or CH3) domains. In some embodiments, the antibody fragment is an antibody fragment of, or derived from, a fully human or humanized antibody. In some embodiments, the antibody fragment is an antibody fragment of, or derived from, a chimeric antibody. The antibody from which the antibody fragment is obtained or derived can be of any of a variety of antibody classes, including, e.g., an IgG1 antibody, an IgG2 antibody, or an IgG4 antibody. In some embodiments, the antibody fragment is obtained or derived from an antagonistic antibody, e.g., an antagonistic humanized antibody.

In embodiments where multiple types of fusion polypeptides having antibody fragments are used, the antibody fragments in the various types of fusion polypeptides may be capable of binding to the same epitope on a given antigen (e.g., an antigen on a cytokine or a tumor-associated antigen), capable of binding to epitopes that are distinct and non-overlapping on an antigen, or capable of binding to epitopes that are distinct but overlapping on the same antigen.

### A. Cytokine-Binding Moieties

Cytokine-binding moieties are generally capable of binding (e.g., specifically binding) to an epitope on a cytokine of interest, e.g., IL-6 and/or IL-11.

In some embodiments, the cytokine-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which are similar to (e.g., each having at most one or two amino acids differing from) those of the heavy and light chain CDRs of an IL-6 or IL-11 antibody (e.g., a human or humanized IL-6 or IL-11 antibody).

In some embodiments, the cytokine-binding moiety is an antibody fragment comprising a heavy chain variable region (V_{H}) and light chain variable region (V_{L} or V_{K}), wherein the CDRs of the V_{H} and the V_{L} or V_{K} have sequences which are identical to those of the heavy and light chain CDRs of an IL-6 or IL-11 antibody (e.g., a human or humanized IL-6 or IL-11 antibody), except for one or two amino acid substitutions total across all six CDRs.

In some embodiments, the cytokine-binding moiety is an antibody fragment comprising a heavy chain variable region (V_{H}) and light chain variable region (V_{L} or V_{K}), wherein the CDRs of the V_{H} and the V_{L} or V_{K} are identical to those of the heavy and light chain CDRs of an IL-6 or IL-11 antibody (e.g., a human or humanized IL-6 or IL-11 antibody).

In some embodiments, the cytokine-binding moiety is an antibody fragment comprising heavy and light chain variable regions (e.g., V_{H} and V_{L} or V_{K}) with sequences that have at least 85%, at least 87.5%, at least 90%, at least 91%, at least 92%, at least 93%, at least 94%, at least 95%, at least 96%, at least 97%, at least 98% or at least 99% identity to those of the heavy and light chain variable regions of a IL-6 or IL-11 antibody (e.g., a human or humanized IL-6 or IL-11 antibody). In some embodiments, the cytokine-binding moiety is an antibody fragment comprising heavy and light chain variable regions (e.g., V_{H} and V_{L} or V_{K}) with sequences identical to those of the heavy and light chain variable regions of an IL-6 or IL-11 antibody (e.g., a human or humanized IL-6 or IL-11 antibody).

Non-limiting examples of IL-6 antibodies include clazakizumab, EBI-029, EBI-031, elsilimomab, MEDI 5117, olokizumab, siltuximab, and sirukumab, whose heavy chain variable region, light chain variable region, and CDR sequences are depicted in Tables 1A-1H.

Non-limiting examples of IL-11 antibodies include 3C6 2.1, 3C6 2.2, 3C6 2.2b, TPP-29536, TPP-30003, YU100-G08, hz8C8m7, and hz18A10m19, whose heavy chain variable region, light chain variable region, and CDR sequences are depicted in Tables 2A-2H.

TABLE 1A
IL-6 Antibody Sequences (Clazakizumab)

| Heavy chain variable region | | Light chain variable region | |
|---|---|---|---|
| EVQLVESGGGLVQPGGSLRLSCAASGFSLSNY | | AIQMTQSPSSLSASVGDRVTITCQASQSIN | |
| YVTWVRQAPGKGLEWVGIIYGSDETAYATSAI | | NELSWYQQKPGKAPKLLIYRASTLASGVPS | |
| GRFTISRDNSKNTLYLQMNSLRAEDTAVYYCA | | RFSGSGSGTDFTLTISSLQPDDFATYYCQQ | |
| RDDSSDWDAKFNLWGQGTLVTVSS | | GYSLRNIDNAFGGGTKVEIKR | |
| (SEQ ID NO: 8) | | (SEQ ID NO: 9) | |
| CDR Sequences (Kabat) | | CDR Sequences (Kabat) | |
| CDR-H1 | NYYVT | CDR-L1 | QASQSINNELS |
| | (SEQ ID NO: 10) | | (SEQ ID NO: 13) |
| CDR-H2 | IIYGSDETAYATSAIG | CDR-L2 | RASTLAS |
| | (SEQ ID NO: 11) | | (SEQ ID NO: 14) |
| CDR-H3 | DDSSDWDAKFNL | CDR-L3 | QQGYSLRNIDNA |
| | (SEQ ID NO: 12) | | (SEQ ID NO: 15) |
| CDR Sequences (IMGT) | | CDR Sequences (IMGT) | |
| CDR-H1 | GFSLSNYY | CDR-L1 | QSINNE |
| | (SEQ ID NO: 16) | | (SEQ ID NO: 19) |
| CDR-H2 | IYGSDET | CDR-L2 | RA |
| | (SEQ ID NO: 17) | CDR-L3 | QQGYSLRNIDNA |
| CDR-H3 | ARDDSSDWDAKFNL | | (SEQ ID NO: 20) |
| | (SEQ ID NO: 18) | | |
| CDR Sequences (Chothia) | | CDR Sequences (Chothia) | |
| CDR-H1 | GFSLSNY | CDR-L1 | QASQSINNELS |
| | (SEQ ID NO: 21) | | (SEQ ID NO: 24) |
| CDR-H2 | YGSDE | CDR-L2 | RASTLAS |
| | (SEQ ID NO: 22) | | (SEQ ID NO: 25) |
| CDR-H3 | DDSSDWDAKFNL | CDR-L3 | QQGYSLRNIDNA |
| | (SEQ ID NO: 23) | | (SEQ ID NO: 26) |

TABLE 1B
IL-6 Antibody Sequences (EBI-029)

| Heavy chain variable region | | Light chain variable region | |
|---|---|---|---|
| QVQLVQSGAEVKKPGSSVKVSCKASGYALSNY | | DIVMTQSPDSLAVSLGERATINCRASESVD | |
| LIEWVRQAPGQGLEWMGVITPGSGTINYAQKF | | NYGIPFMNWYQQKPGQPPKLLIYAASNRGS | |
| QGRVTITADESTSTAYMELSSLRSEDTAVYYC | | GVPDRFSGSGSGTDFTLTISSLQAEDVAVY | |
| ARSRWDPLYYYALEYWGQGTTVTVSS | | YCQQSEEVPLTFGQGTKLEIKR | |
| (SEQ ID NO: 27) | | (SEQ ID NO: 28) | |
| CDR Sequences (Kabat) | | CDR Sequences (Kabat) | |
| CDR-H1 | NYLIE | CDR-L1 | RASESVDNYGIPFMN |
| | (SEQ ID NO: 29) | | (SEQ ID NO: 32) |
| CDR-H2 | VITPGSGTINYAQKFQG | CDR-L2 | AASNRGS |
| | (SEQ ID NO: 30) | | (SEQ ID NO: 33) |
| CDR-H3 | SRWDPLYYYALEY | CDR-L3 | QQSEEVPLT |
| | (SEQ ID NO: 31) | | (SEQ ID NO: 34) |
| CDR Sequences (IMGT) | | CDR Sequences (IMGT) | |
| CDR-H1 | GYALSNYL | CDR-L1 | ESVDNYGIPF |
| | (SEQ ID NO: 35) | | (SEQ ID NO: 38) |
| CDR-H2 | ITPGSGTI | CDR-L2 | AA |
| | (SEQ ID NO: 36) | CDR-L3 | QQSEEVPLT |
| CDR-H3 | ARSRWDPLYYYALEY | | (SEQ ID NO: 39) |
| | (SEQ ID NO: 37) | | |
| CDR Sequences (Chothia) | | CDR Sequences (Chothia) | |
| CDR-H1 | GYALSNY | CDR-L1 | RASESVDNYGIPFMN |
| | (SEQ ID NO: 40) | | (SEQ ID NO: 43) |
| CDR-H2 | TPGSGT | CDR-L2 | AASNRGS |
| | (SEQ ID NO: 41) | | (SEQ ID NO: 44) |
| CDR-H3 | SRWDPLYYYALEY | CDR-L3 | QQSEEVPLT |
| | (SEQ ID NO: 42) | | (SEQ ID NO: 45) |

TABLE 1C
IL-6 Antibody Sequences (EBI-031)

| Heavy chain variable region | | Light chain variable region | |
|---|---|---|---|
| QVQLVQSGAEVKKPGSSVKVSCKASGYVLPNY | | DIVMTQSPDSLAVSLGERATINCRASESVD | |
| LIEWVRQAPGQGLEWMGVTTPGGGTINYAQKF | | NYGIPFMNWYQQKPGQPPKLLIYAASNRGS | |
| QGRVTITADESTSTAYMELSSLRSEDTAVYYC | | GVPDRFSGSGSGTDFTLTISSLQAEDVAVY | |
| ARSRWDPLYYYALEYWGQGTTVTVSS | | YCQQSEEVPLTFGQGTKLEIKR | |
| (SEQ ID NO: 46) | | (SEQ ID NO: 47) | |
| CDR Sequences (Kabat) | | CDR Sequences (Kabat) | |
| CDR-H1 | NYLIE | CDR-L1 | RASESVDNYGIPFMN |
| | (SEQ ID NO: 48) | | (SEQ ID NO: 51) |
| CDR-H2 | VTTPGGGTINYAQKFQG | CDR-L2 | AASNRGS |
| | (SEQ ID NO: 49) | | (SEQ ID NO: 52) |
| CDR-H3 | SRWDPLYYYALEY | CDR-L3 | QQSEEVPLT |
| | (SEQ ID NO: 50) | | (SEQ ID NO: 53) |
| CDR Sequences (IMGT) | | CDR Sequences (IMGT) | |
| CDR-H1 | GYVLPNYL | CDR-L1 | ESVDNYGIPF |
| | (SEQ ID NO: 54) | | (SEQ ID NO: 57) |
| CDR-H2 | TTPGGGTI | CDR-L2 | AA |
| | (SEQ ID NO: 55) | CDR-L3 | QQSEEVPLT |
| CDR-H3 | ARSRWDPLYYYALEY | | (SEQ ID NO: 58) |
| | (SEQ ID NO: 56) | | |
| CDR Sequences (Chothia) | | CDR Sequences (Chothia) | |
| CDR-H1 | GYVLPNY | CDR-L1 | RASESVDNYGIPFMN |
| | (SEQ ID NO: 59) | | (SEQ ID NO: 62) |
| CDR-H2 | TPGGGT | CDR-L2 | AASNRGS |
| | (SEQ ID NO: 60) | | (SEQ ID NO: 63) |
| CDR-H3 | SRWDPLYYYALEY | CDR-L3 | QQSEEVPLT |
| | (SEQ ID NO: 61) | | (SEQ ID NO: 64) |

TABLE 1D
IL-6 Antibody Sequences (Elsilimomab/B-E8)

| Heavy chain variable region | | Light chain variable region | |
|---|---|---|---|
| QVTLKESGPGILKPSQTLSLTCSFSGFSLSTS | | DIVMTQSPATLSVTPGDRVSLSCRASQSIS | |
| GMGVGWIRQPSGKGLEWLAHIWWDDDKYYNPS | | DYLHWYQQKSHESPRLLIKSVSQSISGIPS | |
| LKSQLTISKDTSRNQVFLKITSVDTADTATYY | | RFSGSGSGSDFTLSINSVEPEDVGVYYCQN | |
| CARSYDDYLYYALDYWGQGTSVTVSS | | GHSFPLTFGAGTKLELKR | |
| (SEQ ID NO: 65) | | (SEQ ID NO: 66) | |
| CDR Sequences (Kabat) | | CDR Sequences (Kabat) | |
| CDR-H1 | TSGMGVG | CDR-L1 | RASQSISDYLH |
| | (SEQ ID NO: 67) | | (SEQ ID NO: 70) |
| CDR-H2 | HIWWDDDKYYNPSLKS | CDR-L2 | SVSQSIS |
| | (SEQ ID NO: 68) | | (SEQ ID NO: 71) |
| CDR-H3 | SYDDYLYYALDY | CDR-L3 | QNGHSFPLT |
| | (SEQ ID NO: 69) | | (SEQ ID NO: 72) |
| CDR Sequences (IMGT) | | CDR Sequences (IMGT) | |
| CDR-H1 | GFSLSTSGMG | CDR-L1 | QSISDY |
| | (SEQ ID NO: 73) | | (SEQ ID NO: 76) |
| CDR-H2 | IWWDDDK | CDR-L2 | SV |
| | (SEQ ID NO: 74) | CDR-L3 | QNGHSFPLT |
| CDR-H3 | ARSYDDYLYYALDY | | (SEQ ID NO: 77) |
| | (SEQ ID NO: 75) | | |
| CDR Sequences (Chothia) | | CDR Sequences (Chothia) | |
| CDR-H1 | GFSLSTSGM | CDR-L1 | RASQSISDYLH |
| | (SEQ ID NO: 78) | | (SEQ ID NO: 81) |
| CDR-H2 | WWDDD | CDR-L2 | SVSQSIS |
| | (SEQ ID NO: 79) | | (SEQ ID NO: 82) |
| CDR-H3 | SYDDYLYYALDY | CDR-L3 | QNGHSFPLT |
| | (SEQ ID NO: 80) | | (SEQ ID NO: 83) |

TABLE 1E
IL-6 Antibody Sequences (MEDI 5117)

| | |
|---|---|
| Heavy chain variable region | Light chain variable region |
| EVQLVESGGGLVQPGGSLRLSCAASGFTISSN | DIQMTQSPSTLSASVGDRVTITCRASQGIS |
| YMIWVRQAPGKGLEWVSDLYYYAGDTYYADSV | SWLAWYQQKPGKAPKVLIYKASTLESGVPS |
| KGRFTMSRDISKNTVYLQMNSLRAEDTAVYYC | RFSGSGSGTEFTLTISSLQPDDFATYYCQQ |
| ARWADDHPPWIDLWGRGTLVTVSS | SWLGGSFGQGTKLEIKR |
| (SEQ ID NO: 84) | (SEQ ID NO: 85) |
| CDR Sequences (Kabat) | CDR Sequences (Kabat) |
| CDR-H1 SNYMI | CDR-L1 RASQGISSWLA |
| (SEQ ID NO: 86) | (SEQ ID NO: 89) |
| CDR-H2 DLYYYAGDTYYADSVKG | CDR-L2 KASTLES |
| (SEQ ID NO: 87) | (SEQ ID NO: 90) |
| CDR-H3 WADDHPPWIDL | CDR-L3 QQSWLGGS |
| (SEQ ID NO: 88) | (SEQ ID NO: 91) |
| CDR Sequences (IMGT) | CDR Sequences (IMGT) |
| CDR-H1 GFTISSNY | CDR-L1 QGISSW |
| (SEQ ID NO: 92) | (SEQ ID NO: 95) |
| CDR-H2 LYYYAGDT | CDR-L2 KA |
| (SEQ ID NO: 93) | CDR-L3 QQSWLGGS |
| CDR-H3 ARWADDHPPWIDL | (SEQ ID NO: 96) |
| (SEQ ID NO: 94) | |
| CDR Sequences (Chothia) | CDR Sequences (Chothia) |
| CDR-H1 GFTISSN | CDR-L1 RASQGISSWLA |
| (SEQ ID NO: 97) | (SEQ ID NO: 100) |
| CDR-H2 YYYAGD | CDR-L2 KASTLES |
| (SEQ ID NO: 98) | (SEQ ID NO: 101) |
| CDR-H3 WADDHPPWIDL | CDR-L3 QQSWIGGS |
| (SEQ ID NO: 99) | (SEQ ID NO: 102) |

TABLE 1F
IL-6 Antibody Sequences (Olokizumab/CDP6038)

| | |
|---|---|
| Heavy chain variable region | Light chain variable region |
| EVQLVESGGGLVQPGGSLRLSCAASGENENDY | DIQMTQSPSSLSASVGDRVTITCQASQDIG |
| FMNWVRQAPGKGLEWVAQMRNKNYQYGTYYAE | ISLSWYQQKPGKAPKLLIYNANNLADGVPS |
| SLEGRFTISRDDSKNSLYLQMNSLKTEDTAVY | RFSGSGSGTDFTLTISSLQPEDFATYYCLQ |
| YCARESYYGFTSYWGQGTLVTVSS | HNSAPYTFGQGTKLEIKR |
| (SEQ ID NO: 103) | (SEQ ID NO: 104) |
| CDR Sequences (Kabat) | CDR Sequences (Kabat) |
| CDR-H1 DYFMN | CDR-L1 QASQDIGISLS |
| (SEQ ID NO: 105) | (SEQ ID NO: 108) |
| CDR-H2 QMRNKNYQYGTYYAESLEG | CDR-L2 NANNLAD |
| (SEQ ID NO: 106) | (SEQ ID NO: 109) |
| CDR-H3 ESYYGFTSY | CDR-L3 LQHNSAPYT |
| (SEQ ID NO: 107) | (SEQ ID NO: 110) |
| CDR Sequences (IMGT) | CDR Sequences (IMGT) |
| CDR-H1 GENENDYF | CDR-L1 QDIGIS |
| (SEQ ID NO: 111) | (SEQ ID NO: 114) |
| CDR-H2 MRNKNYQYGT | CDR-L2 NA |
| (SEQ ID NO: 112) | CDR-L3 LQHNSAPYT |
| CDR-H3 ARESYYGFTSY | (SEQ ID NO: 115) |
| (SEQ ID NO: 113) | |
| CDR Sequences (Chothia) | CDR Sequences (Chothia) |
| CDR-H1 GENENDY | CDR-L1 QASQDIGISLS |
| (SEQ ID NO: 116) | (SEQ ID NO: 119) |
| CDR-H2 RNKNYQYG | CDR-L2 NANNLAD |
| (SEQ ID NO: 117) | (SEQ ID NO: 120) |
| CDR-H3 ESYYGFTSY | CDR-L3 LQHNSAPYT |
| (SEQ ID NO: 118) | (SEQ ID NO: 121) |

TABLE 1G
IL-6 Antibody Sequences (Siltuximab/CNTO-328)

| | |
|---|---|
| Heavy chain variable region | Light chain variable region |
| EVQLVESGGKLLKPGGSLKLSCAASGFTFSSF | QIVLIQSPAIMSASPGEKVTMTCSASSSVS |
| AMSWFRQSPEKRLEWVAEISSGGSYTYYPDTV | YMYWYQQKPGSSPRLLIYDTSNLASGVPVR |
| TGRFTISRDNAKNTLYLEMSSLRSEDTAMYYC | FSGSGSGTSYSLTISRMEAEDAATYYCQQW |
| ARGLWGYYALDYWGQGTSVTVSS | SGYPYTFGGGTKLEIKR |
| (SEQ ID NO: 122) | (SEQ ID NO: 123) |
| CDR Sequences (Kabat) | CDR Sequences (Kabat) |
| CDR-H1 S FAMS | CDR-L1 SASSSVSYMY |
| (SEQ ID NO: 124) | (SEQ ID NO: 127) |
| CDR-H2 EISSGGSYTYYPDTVTG | CDR-L2 DTSNLAS |
| (SEQ ID NO: 125) | (SEQ ID NO: 128) |
| CDR-H3 GLWGYYALDY | CDR-L3 QQWSGYPYT |
| (SEQ ID NO: 126) | (SEQ ID NO: 129) |
| CDR Sequences (IMGT) | CDR Sequences (IMGT) |
| CDR-H1 GFTESSFA | CDR-L1 SSVSY |
| (SEQ ID NO: 130) | (SEQ ID NO: 133) |
| CDR-H2 ISSGGSYT | CDR-L2 DT |
| (SEQ ID NO: 131) | CDR-L3 QQWSGYPYT |
| CDR-H3 ARGLWGYYALDY | (SEQ ID NO: 134) |
| (SEQ ID NO: 132) | |
| CDR Sequences (Chothia) | CDR Sequences (Chothia) |
| CDR-H1 GFTFSSF | CDR-L1 SASSSVSYMY |
| (SEQ ID NO: 135) | (SEQ ID NO: 138) |
| CDR-H2 SSGGSY | CDR-L2 DTSNLAS |
| (SEQ ID NO: 136) | (SEQ ID NO: 139) |
| CDR-H3 GLWGYYALDY | CDR-L3 QQWSGYPYT |
| (SEQ ID NO: 137) | (SEQ ID NO: 140) |

TABLE 1H
IL-6 Antibody Sequences (Sirukumab/CNTO 136)

| | |
|---|---|
| Heavy chain variable region | Light chain variable region |
| EVQLVESGGGLVQPGGSLRLSCAASGFTFSPF | EIVLTQSPATLSLSPGERATLSCSASISVS |
| AMSWVRQAPGKGLEWVAKISPGGSWTYYSDTV | YMYWYQQKPGQAPRLLIYDMSNLASGIPAR |
| TGRFTISRDNAKNSLYLQMNSLRAEDTAVYYC | FSGSGSGTDFTLTISSLEPEDFAVYYCMQW |
| ARQLWGYYALDIWGQGTTVTVSS | SGYPYTFGGGTKVEIKR |
| (SEQ ID NO: 141) | (SEQ ID NO: 142) |
| CDR Sequences (Kabat) | CDR Sequences (Kabat) |
| CDR-H1 PFAMS | CDR-L1 SASISVSYMY |
| (SEQ ID NO: 143) | (SEQ ID NO: 146) |
| CDR-H2 KISPGGSWTYYSDTVTG | CDR-L2 DMSNLAS |
| (SEQ ID NO: 144) | (SEQ ID NO: 147) |
| CDR-H3 QLWGYYALDI | CDR-L3 MQWSGYPYT |
| (SEQ ID NO: 145) | (SEQ ID NO: 148) |
| CDR Sequences (IMGT) | CDR Sequences (IMGT) |
| CDR-H1 GFTESPFA | CDR-L1 ISVSY |
| (SEQ ID NO: 149) | (SEQ ID NO: 152) |
| CDR-H2 ISPGGSWT | CDR-L2 DM |
| (SEQ ID NO: 150) | CDR-L3 MQWSGYPYT |
| CDR-H3 ARQLWGYYALDI | (SEQ ID NO: 153) |
| (SEQ ID NO: 151) | |
| CDR Sequences (Chothia) | CDR Sequences (Chothia) |
| CDR-H1 GFTFSPF | CDR-L1 SASISVSYMY |
| (SEQ ID NO: 154) | (SEQ ID NO: 157) |
| CDR-H2 SPGGSW | CDR-L2 DMSNLAS |
| (SEQ ID NO: 155) | (SEQ ID NO: 158) |
| CDR-H3 QLWGYYALDI | CDR-L3 MQWSGYPYT |
| (SEQ ID NO: 156) | (SEQ ID NO: 159) |

TABLE 2A
IL-11 Antibody Sequences (3C6 2.1)

| | |
|---|---|
| Heavy chain variable region | Light chain variable region |
| EVQLVQSGAEVKKPGASVKISCKASGYTFTDY | DIVLTQSPASLALSPGERATLSCRASKSVS |
| NMDWVKQAPGQRLEWIGDINPHNGGPIYNQKF | TSGYSYIHWYQQKPGQPPRLLIYLASNLDS |
| TGRATLTVDKSASTAYMELSSLRSEDTAVYYC | GVPARFSGSGSGTDFTLNIHPLEEEDFATY |
| ARGELGHWYFDVWGQGTTVTVSS (SEQ ID | YCQHSRDLPPTFGQGTKLEIK |
| NO: 160) | (SEQ ID NO: 161) |
| CDR Sequences (Kabat) | CDR Sequences (Kabat) |
| CDR-H1 DYNMD | CDR-L1 RASKSVSTSGYSYIH |
| (SEQ ID NO: 162) | (SEQ ID NO: 165) |
| CDR-H2 DINPHNGGPIYNQKFTG | CDR-L2 LASNLDS |
| (SEQ ID NO: 163) | (SEQ ID NO: 166) |
| CDR-H3 GELGHWYFDV | CDR-L3 QHSRDLPPT |
| (SEQ ID NO: 164) | (SEQ ID NO: 167) |
| CDR Sequences (IMGT) | CDR Sequences (IMGT) |
| CDR-H1 GYTFTDYN | CDR-L1 KSVSTSGYSY |
| (SEQ ID NO: 168) | (SEQ ID NO: 171) |
| CDR-H2 INPHNGGP | CDR-L2 LA |
| (SEQ ID NO: 169) | CDR-L3 QHSRDLPPT |
| CDR-H3 ARGELGHWYFDV | (SEQ ID NO: 172) |
| (SEQ ID NO: 170) | |
| CDR Sequences (Chothia) | CDR Sequences (Chothia) |
| CDR-H1 GYTFTDY | CDR-L1 RASKSVSTSGYSYIH |
| (SEQ ID NO: 173) | (SEQ ID NO: 176) |
| CDR-H2 NPHNGG | CDR-L2 LASNLDS |
| (SEQ ID NO: 174) | (SEQ ID NO: 177) |
| CDR-H3 GELGHWYFDV | CDR-L3 QHSRDLPPT |
| (SEQ ID NO: 175) | (SEQ ID NO: 178) |

TABLE 2B
IL-11 Antibody Sequences (3C6 2.2)

| | |
|---|---|
| Heavy chain variable region | Light chain variable region |
| EVQLVQSGAEVKKPGASVKISCKASGYTFTDY | DIVLTQSPASLALSPGERATLSCRASKSVS |
| NMDWVKQAPGQRLEWIGDINPHNGGPIYNQKF | TSGYSYIHWYQQKPGQAPRLLIYLASNLDS |
| TGRATLTVDKSASTAYMELSSLRSEDTAVYYC | GVPARFSGSGSGTDFTLTISSLEEEDFATY |
| ARGELGHWYFDVWGQGTTVTVSS | YCQHSRDLPPTFGQGTKLEIK |
| (SEQ ID NO: 179) | (SEQ ID NO: 180) |
| CDR Sequences (Kabat) | CDR Sequences (Kabat) |
| CDR-H1 DYNMD | CDR-L1 RASKSVSTSGYSYIH |
| (SEQ ID NO: 181) | (SEQ ID NO: 184) |
| CDR-H2 DINPHNGGPIYNQKFTG | CDR-L2 LASNLDS |
| (SEQ ID NO: 182) | (SEQ ID NO: 185) |
| CDR-H3 GELGHWYFDV | CDR-L3 QHSRDLPPT |
| (SEQ ID NO: 183) | (SEQ ID NO: 186) |
| CDR Sequences (IMGT) | CDR Sequences (IMGT) |
| CDR-H1 GYTFTDYN | CDR-L1 KSVSTSGYSY |
| (SEQ ID NO: 187) | (SEQ ID NO: 190) |
| CDR-H2 INPHNGGP | CDR-L2 LA |
| (SEQ ID NO: 188) | CDR-L3 QHSRDLPPT |
| CDR-H3 ARGELGHWYFDV | (SEQ ID NO: 191) |
| (SEQ ID NO: 189) | |
| CDR Sequences (Chothia) | CDR Sequences (Chothia) |
| CDR-H1 GYTFTDY | CDR-L1 RASKSVSTSGYSYIH |
| (SEQ ID NO: 192) | (SEQ ID NO: 195) |
| CDR-H2 NPHNGG | CDR-L2 LASNLDS |
| (SEQ ID NO: 193) | (SEQ ID NO: 196) |
| CDR-H3 GELGHWYFDV | CDR-L3 QHSRDLPPT |
| (SEQ ID NO: 194) | (SEQ ID NO: 197) |

TABLE 2C
IL-11 Antibody Sequences (3C6 2.2b)

| | |
|---|---|
| Heavy chain variable region | Light chain variable region |
| EVQLVQSGAEVKKPGASVKISCKASGYTFTDY | DIVLTQSPASLALSPGERATLSCRASKSVS |
| NMDWVKQAPGQRLEWIGDINPHNGGPIYNQKF | TSGYSYIHWYQQKPGQPPRLLIYLASNLDS |
| TGRATLTVDKSASTAYMELSSLRSEDTAVYYC | GVPARFSGSGSGTDFTLNIHPLEEEDFATY |
| ARGELGHWYFDVWGQGTTVTVSS | YCQHSRDLPPTFGQGTKLEIK |
| (SEQ ID NO: 198) | (SEQ ID NO: 199) |
| CDR Sequences (Kabat) | CDR Sequences (Kabat) |
| CDR-H1 DYNMD | CDR-L1 RASKSVSTSGYSYIH |
| (SEQ ID NO: 200) | (SEQ ID NO: 203) |
| CDR-H2 DINPHNGGPIYNQKFTG | CDR-L2 LASNLDS |
| (SEQ ID NO: 201) | (SEQ ID NO: 204) |
| CDR-H3 GELGHWYFDV | CDR-L3 QHSRDLPPT |
| (SEQ ID NO: 202) | (SEQ ID NO: 205) |
| CDR Sequences (IMGT) | CDR Sequences (IMGT) |
| CDR-H1 GYTFTDYN | CDR-L1 KSVSTSGYSY |
| (SEQ ID NO: 206) | (SEQ ID NO: 209) |
| CDR-H2 INPHNGGP | CDR-L2 LA |
| (SEQ ID NO: 207) | CDR-L3 QHSRDLPPT |
| CDR-H3 ARGELGHWYFDV | (SEQ ID NO: 210) |
| (SEQ ID NO: 208) | |
| CDR Sequences (Chothia) | CDR Sequences (Chothia) |
| CDR-H1 GYTFTDY | CDR-L1 RASKSVSTSGYSYIH |
| (SEQ ID NO: 211) | (SEQ ID NO: 214) |
| CDR-H2 NPHNGG | CDR-L2 LASNLDS |
| (SEQ ID NO: 212) | (SEQ ID NO: 215) |
| CDR-H3 GELGHWYFDV | CDR-L3 QHSRDLPPT |
| (SEQ ID NO: 213) | (SEQ ID NO: 216) |

TABLE 2D
IL-11 Antibody Sequences (TPP-29536)

| | |
|---|---|
| Heavy chain variable region | Light chain variable region |
| EVQLLESGGGLVQPGGSLRLSCAASGFTESSY | QSVLTQPPSASGTPGQRVTISCSGSSSNIG |
| GMHWVRQAPGKGLEWVAVISYDGSYKYYADSV | SDYDVHWYQQLPGTAPKLLIYSNEERPSGV |
| KGRFTISRDNSKNTLYLQMNSLRAEDTAVYYC | PDRFSGSKSGTSASLAISGLRSEDEADYYC |
| AAGVPDYWGQGTLVTVSS | SAWDSSLSGPVEGGGTKLTVL |
| (SEQ ID NO: 217) | (SEQ ID NO: 218) |
| CDR Sequences (Kabat) | CDR Sequences (Kabat) |
| CDR-H1 SYGMH | CDR-L1 SGSSSNIGSDYDVH |
| (SEQ ID NO: 219) | (SEQ ID NO: 222) |
| CDR-H2 VISYDGSYKYYADSVKG | CDR-L2 SNEERPS |
| (SEQ ID NO: 220) | (SEQ ID NO: 223) |
| CDR-H3 GVPDY | CDR-L3 SAWDSSLSGPV |
| (SEQ ID NO: 221) | (SEQ ID NO: 224) |
| CDR Sequences (IMGT) | CDR Sequences (IMGT) |
| CDR-H1 GFTFSSYG | CDR-L1 SSNIGSDYD |
| (SEQ ID NO: 225) | (SEQ ID NO: 228) |
| CDR-H2 ISYDGSYK | CDR-L2 SN |
| (SEQ ID NO: 226) | CDR-L3 SAWDSSLSGPV |
| CDR-H3 AAGVPDY | (SEQ ID NO: 229) |
| (SEQ ID NO: 227) | |
| CDR Sequences (Chothia) | CDR Sequences (Chothia) |
| CDR-H1 GFTFSSY | CDR-L1 SGSSSNIGSDYDVH |
| (SEQ ID NO: 230) | (SEQ ID NO: 233) |
| CDR-H2 SYDGSY | CDR-L2 SNEERPS |
| (SEQ ID NO: 231) | (SEQ ID NO: 234) |
| CDR-H3 GVPDY | CDR-L3 SAWDSSLSGPV |
| (SEQ ID NO: 232) | (SEQ ID NO: 235) |

TABLE 2E
IL-11 Antibody Sequences (TPP-30003)

| | |
|---|---|
| Heavy chain variable region | Light chain variable region |
| QVQLVESGGGVVQPGRSLRLSCAASGFTESHY | QSVLTQPPSASGTPGQRVTISCSGSSSNIG |
| DMHWVRQAPGKGLEWVAVISYEGSVKFYADSV | SNYVNWYQQLPGTAPKLLIYYDDLRPSGVP |
| KGRFTISRDNSKNTLYLQMNSLRAEDTAVYYC | DRFSGSKSGTSASLAISGLRSEDEADYYCS |
| ARTPGFHFDYWGQGTLVTVSS | AWDDSLSGVVFGGGTKLTVL |
| (SEQ ID NO: 236) | (SEQ ID NO: 237) |
| CDR Sequences (Kabat) | CDR Sequences (Kabat) |
| CDR-H1 HYDMH | CDR-L1 SGSSSNIGSNYVN |
| (SEQ ID NO: 238) | (SEQ ID NO: 241) |
| CDR-H2 VISYEGSVKFYADSVKG | CDR-L2 YDDLRPS |
| (SEQ ID NO: 239) | (SEQ ID NO: 242) |
| CDR-H3 TPGFHFDY | CDR-L3 SAWDDSLSGVV |
| (SEQ ID NO: 240) | (SEQ ID NO: 243) |
| CDR Sequences (IMGT) | CDR Sequences (IMGT) |
| CDR-H1 GFTFSHYD | CDR-L1 SSNIGSNY |
| (SEQ ID NO: 244) | (SEQ ID NO: 247) |
| CDR-H2 ISYEGSVK | CDR-L2 YD |
| (SEQ ID NO: 245) | CDR-L3 SAWDDSLSGVV |
| CDR-H3 ARTPGFHFDY | (SEQ ID NO: 248) |
| (SEQ ID NO: 246) | |
| CDR Sequences (Chothia) | CDR Sequences (Chothia) |
| CDR-H1 GFTFSHY | CDR-L1 SGSSSNIGSNYVN |
| (SEQ ID NO: 249) | (SEQ ID NO: 252) |
| CDR-H2 SYEGSV | CDR-L2 YDDLRPS |
| (SEQ ID NO: 250) | (SEQ ID NO: 253) |
| CDR-H3 TPGFHFDY | CDR-L3 SAWDDSLSGVV |
| (SEQ ID NO: 251) | (SEQ ID NO: 254) |

TABLE 2F
IL-11 Antibody Sequences (YU100-G08)

| | |
|---|---|
| Heavy chain variable region | Light chain variable region |
| QVQLVQSGGGVVQPGRSLRLSCAASGFTFSSY | QSALTQPRSVSGSPGQSVTLSCTGTSSDVG |
| GMHWVRQAPGKGLEWVAVISYDGSNKYYADSV | GYNYVSWYQHYPGKAPKLMIFDVNERSSGV |
| KGRFTISRDNSKNTLYLQMNSLRAEDTAVYYC | PDRFSGSKSGNTASLTISGLQAEDEADYYC |
| AKIGATDPLDYWGQGTLVTVSS | CSYAGRYTWMFGGGTKVTVLG |
| (SEQ ID NO: 255) | (SEQ ID NO: 256) |
| CDR Sequences (Kabat) | CDR Sequences (Kabat) |
| CDR-H1 SYGMH | CDR-L1 TGTSSDVGGYNYVS |
| (SEQ ID NO: 257) | (SEQ ID NO: 260) |
| CDR-H2 VISYDGSNKYYADSVKG | CDR-L2 DVNERSS |
| (SEQ ID NO: 258) | (SEQ ID NO: 261) |
| CDR-H3 IGATDPLDY | CDR-L3 CSYAGRYTWM |
| (SEQ ID NO: 259) | (SEQ ID NO: 262) |
| CDR Sequences (IMGT) | CDR Sequences (IMGT) |
| CDR-H1 GFTFSSYG | CDR-L1 SSDVGGYNY |
| (SEQ ID NO: 263) | (SEQ ID NO: 266) |
| CDR-H2 ISYDGSNK | CDR-L2 DV |
| (SEQ ID NO: 264) | CDR-L3 CSYAGRYTWM |
| CDR-H3 AKIGATDPLDY | (SEQ ID NO: 267) |
| (SEQ ID NO: 265) | |
| CDR Sequences (Chothia) | CDR Sequences (Chothia) |
| CDR-H1 GFTESSY | CDR-L1 TGTSSDVGGYNYVS |
| (SEQ ID NO: 268) | (SEQ ID NO: 271) |
| CDR-H2 SYDGSN | CDR-L2 DVNERSS |
| (SEQ ID NO: 269) | (SEQ ID NO: 272) |
| CDR-H3 IGATDPLDY | CDR-L3 CSYAGRYTWM |
| (SEQ ID NO: 270) | (SEQ ID NO: 273) |

TABLE 2G
IL-11 Antibody Sequences (hz8C8m7)

| | |
|---|---|
| Heavy chain variable region | Light chain variable region |
| EVQLVESGGGLVQPGGSLRLSCAASGFTFSSF | EIVLTQSPATLSLSPGERATLSCSVSDLIP |
| GMHWVRQAPGKGLEWVAYITPGSNTIYYADTV | SQYLHWYQQKPGQAPRLLIYRTSNLASGIP |
| KGRFTISRDNAKNSLYLQMNSLRAEDTAVYYC | ARFSGSGSGTDFTLTISSLEPEDFAVYYCQ |
| AREYYSYDEGFAYWGQGTLVTVSS | QGVDVPFTFGQGTKLEIK |
| (SEQ ID NO: 274) | (SEQ ID NO: 275) |
| CDR Sequences (Kabat) | CDR Sequences (Kabat) |
| CDR-H1 SFGMH | CDR-L1 SVSDLIPSQYLH |
| (SEQ ID NO: 276) | (SEQ ID NO: 279) |
| CDR-H2 YITPGSNTIYYADTVKG | CDR-L2 RTSNLAS |
| (SEQ ID NO: 277) | (SEQ ID NO: 280) |
| CDR-H3 EYYSYDEGFAY | CDR-L3 QQGVDVPFT |
| (SEQ ID NO: 278) | (SEQ ID NO: 281) |
| CDR Sequences (IMGT) | CDR Sequences (IMGT) |
| CDR-H1 GFTFSSFG | CDR-L1 DLIPSQY |
| (SEQ ID NO: 282) | (SEQ ID NO: 285) |
| CDR-H2 ITPGSNTI | CDR-L2 RTS |
| (SEQ ID NO: 283) | CDR-L3 QQGVDVPFT |
| CDR-H3 AREYYSYDEGFAY | (SEQ ID NO: 286) |
| (SEQ ID NO: 284) | |
| CDR Sequences (Chothia) | CDR Sequences (Chothia) |
| CDR-H1 GFTFSSF | CDR-L1 SVSDLIPSQYLH |
| (SEQ ID NO: 287) | (SEQ ID NO: 290) |
| CDR-H2 TPGSNT | CDR-L2 RTSNLAS |
| (SEQ ID NO: 288) | (SEQ ID NO: 291) |
| CDR-H3 EYYSYDEGFAY | CDR-L3 QQGVDVPFT |
| (SEQ ID NO: 289) | (SEQ ID NO: 292) |

TABLE 2H
IL-11 Antibody Sequences (hz18A10m19)

| | |
|---|---|
| Heavy chain variable region | Light chain variable region |
| EVQLVQSGAEVKKPGASVKVSCKASGYTFTHY | DIQMTQSPSSLSASVGDRVTITCKASQSVY |
| NMDWVRQAPGQGLEWMGTIYPGNADTSYNQKF | RDVAWYQQKPGKAPKLLIYSASNRYTGVPS |
| KGRVTMTRDTSTSTVYMELSSLRSEDTAVYYC | RFSGSGSGTDFTFTISSLQPEDIATYYCQH |
| ARGDLYALDYWGQGTLVTVSS | DYSSPYTFGQGTKLEIK |
| (SEQ ID NO: 293) | (SEQ ID NO: 294) |
| CDR Sequences (Kabat) | CDR Sequences (Kabat) |
| CDR-H1 HYNMD | CDR-L1 KASQSVYRDVA |
| (SEQ ID NO: 295) | (SEQ ID NO: 298) |
| CDR-H2 TIYPGNADTSYNQKFKG | CDR-L2 SASNRYT |
| (SEQ ID NO: 296) | (SEQ ID NO: 299) |
| CDR-H3 GDLYALDY | CDR-L3 QHDYSSPYT |
| (SEQ ID NO: 297) | (SEQ ID NO: 300) |
| CDR Sequences (IMGT) | CDR Sequences (IMGT) |
| CDR-H1 GYTFTHYN | CDR-L1 QSVYRD |
| (SEQ ID NO: 301) | (SEQ ID NO: 304) |
| CDR-H2 IYPGNADT | CDR-L2 SAS |
| (SEQ ID NO: 302) | CDR-L3 QHDYSSPYT |
| CDR-H3 ARGDLYALDY | (SEQ ID NO: 305) |
| (SEQ ID NO: 303) | |
| CDR Sequences (Chothia) | CDR Sequences (Chothia) |
| CDR-H1 GYTFTHY | CDR-L1 KASQSVYRDVA |
| (SEQ ID NO: 306) | (SEQ ID NO: 309) |
| CDR-H2 YPGNAD | CDR-L2 SASNRYT |
| (SEQ ID NO: 307) | (SEQ ID NO: 310) |
| CDR-H3 GDLYALDY | CDR-L3 QHDYSSPYT |
| (SEQ ID NO: 308) | (SEQ ID NO: 311) |

### B. Tumor-Binding Moieties

Tumor-binding moieties are generally capable of binding (e.g., specifically binding) to a tumor-associated antigen (e.g., a tumor-specific antigen) expressed on a tumor cell or a cell that supports a tumor (e.g., a stromal cell). In some embodiments, the tumor-binding moiety is capable of binding (e.g., specifically binding) to PD-L1.

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which are similar to (e.g., each having at most one or two amino acids differing from) the CDRs of a PD-L1 antibody (e.g., a human or humanized PD-L1 antibody).

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} have sequences which are identical to those of the heavy and light chain CDRs of a PD-L1 antibody (e.g., a human or humanized PD-L1 antibody), except for one or two amino acid substitutions total across all six CDRs.

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} are identical to those of the heavy and light chain CDRs of a PD-L1 antibody (e.g., a human or humanized PD-L1 antibody).

In some embodiments, the tumor-binding moiety comprises an antibody fragment comprising heavy and light chain variable regions (e.g., V_{H} and V_{L} or V_{K}) with sequences that have at least 85%, at least 87.5%, at least 90%, at least 91%, at least 92%, at least 93%, at least 94%, at least 95%, at least 96%, at least 97%, at least 98% or at least 99% identity to those of the heavy and light chain variable regions of a PD-L1 antibody, e.g., a human or humanized PD-L1 antibody. In some embodiments, the tumor-binding moiety is an antibody fragment comprising heavy and light chain variable regions (e.g., V_{H} and V_{L} or V_{K}) with sequences identical to those of the heavy and light chain variable regions of a PD-L1 antibody (e.g., a human or humanized PD-L1 antibody).

Non-limiting examples of PD-L1 antibodies include atezolizumab, avelumab, and durvalumab, whose heavy chain variable region, light chain variable region, and CDR sequences are depicted in Tables 3A-3C.

TABLE 3A
PD-L1 Antibody Sequences (Atezolizumab)

| | |
|---|---|
| Heavy chain variable region | Light chain variable region |
| EVQLVESGGGLVQPGGSLRLSCAASGFTFSDS | DIQMTQSPSSLSASVGDRVTITCRASQDVS |
| WIHWVRQAPGKGLEWVAWISPYGGSTYYADSV | TAVAWYQQKPGKAPKLLIYSASFLYSGVPS |
| KGRFTISADTSKNTAYLQMNSLRAEDTAVYYC | RFSGSGSGTDFTLTISSLQPEDFATYYCQQ |
| ARRHWPGGFDYWGQGTLVTVSS | YLYHPATFGQGTKVEIKR |
| (SEQ ID NO: 312) | (SEQ ID NO: 313) |
| CDR Sequences (Kabat) | CDR Sequences (Kabat) |
| CDR-H1 DSWIH | CDR-L1 RASQDVSTAVA |
| (SEQ ID NO: 314) | (SEQ ID NO: 317) |
| CDR-H2 WISPYGGSTYYADSVKG | CDR-L2 SASFLYS |
| (SEQ ID NO: 315) | (SEQ ID NO: 318) |
| CDR-H3 RHWPGGFDY | CDR-L3 QQYLYHPAT |
| (SEQ ID NO: 316) | (SEQ ID NO: 319) |
| CDR Sequences (IMGT) | CDR Sequences (IMGT) |
| CDR-H1 GFTFSDSW | CDR-L1 QDVSTA |
| (SEQ ID NO: 320) | (SEQ ID NO: 323) |
| CDR-H2 ISPYGGST | CDR-L2 SA |
| (SEQ ID NO: 321) | CDR-L3 QQYLYHPAT |
| CDR-H3 ARRHWPGGFDY | (SEQ ID NO: 324) |
| (SEQ ID NO: 322) | |
| CDR Sequences (Chothia) | CDR Sequences (Chothia) |
| CDR-H1 GFTESDS | CDR-L1 RASQDVSTAVA |
| (SEQ ID NO: 325) | (SEQ ID NO: 328) |
| CDR-H2 SPYGGS | CDR-L2 SASFLYS |
| (SEQ ID NO: 326) | (SEQ ID NO: 329) |
| CDR-H3 RHWPGGFDY | CDR-L3 QQYLYHPAT |
| (SEQ ID NO: 327) | (SEQ ID NO: 330) |

TABLE 3B
PD-L1 Antibody Sequences (Avelumab)

| | |
|---|---|
| Heavy chain variable region | Light chain variable region |
| EVQLLESGGGLVQPGGSLRLSCAASGFTESSY | QSALTQPASVSGSPGQSITISCTGTSSDVG |
| IMMWVRQAPGKGLEWVSSIYPSGGITFYADTV | GYNYVSWYQQHPGKAPKLMIYDVSNRPSGV |
| KGRFTISRDNSKNTLYLQMNSLRAEDTAVYYC | SNRFSGSKSGNTASLTISGLQAEDEADYYC |
| ARIKLGTVTTVDYWGQGTLVTVSS | SSYTSSSTRVFGTGTKVTVLGQP |
| (SEQ ID NO: 331) | (SEQ ID NO: 332) |
| CDR Sequences (Kabat) | CDR Sequences (Kabat) |
| CDR-H1 SYIMM | CDR-L1 TGTSSDVGGYNYVS |
| (SEQ ID NO: 333) | (SEQ ID NO: 336) |
| CDR-H2 SIYPSGGITFYADTVKG | CDR-L2 DVSNRPS |
| (SEQ ID NO: 334) | (SEQ ID NO: 337) |
| CDR-H3 IKLGTVTTVDY | CDR-L3 SSYTSSSTRV |
| (SEQ ID NO: 335) | (SEQ ID NO: 338) |
| CDR Sequences (IMGT) | CDR Sequences (IMGT) |
| CDR-H1 GFTFSSYI | CDR-L1 SSDVGGYNY |
| (SEQ ID NO: 339) | (SEQ ID NO: 342) |
| CDR-H2 IYPSGGIT | CDR-L2 DV |
| (SEQ ID NO: 340) | CDR-L3 SSYTSSSTRV |
| CDR-H3 ARIKLGTVTTVDY | (SEQ ID NO: 343) |
| (SEQ ID NO: 341) | |
| CDR Sequences (Chothia) | CDR Sequences (Chothia) |
| CDR-H1 GFTFSSY | CDR-L1 TGTSSDVGGYNYVS |
| (SEQ ID NO: 344) | (SEQ ID NO: 347) |
| CDR-H2 YPSGGI | CDR-L2 DVSNRPS |
| (SEQ ID NO: 345) | (SEQ ID NO: 348) |
| CDR-H3 IKLGTVTTVDY | CDR-L3 SSYTSSSTRV |
| (SEQ ID NO: 346) | (SEQ ID NO: 349) |

TABLE 3C
PD-L1 Antibody Sequences (Durvalumab)

| | |
|---|---|
| Heavy chain variable region | Light chain variable region |
| EVQLVESGGGLVQPGGSLRLSCAASGFTFSRY | EIVLTQSPGTLSLSPGERATLSCRASQRVS |
| WMSWVRQAPGKGLEWVANIKQDGSEKYYVDSV | SSYLAWYQQKPGQAPRLLIYDASSRATGIP |
| KGRFTISRDNAKNSLYLQMNSLRAEDTAVYYC | DRFSGSGSGTDFTLTISRLEPEDFAVYYCQ |
| AREGGWFGELAFDYWGQGTLVTVSS | QYGSLPWTFGQGTKVEIKRTV |
| (SEQ ID NO: 350) | (SEQ ID NO: 351) |
| CDR Sequences (Kabat) | CDR Sequences (Kabat) |
| CDR-H1 RYWMS | CDR-L1 RASQRVSSSYLA |
| (SEQ ID NO: 352) | (SEQ ID NO: 355) |
| CDR-H2 NIKQDGSEKYYVDSVKG | CDR-L2 DASSRAT |
| (SEQ ID NO: 353) | (SEQ ID NO: 356) |
| CDR-H3 EGGWFGELAFDY | CDR-L3 QQYGSLPWT |
| (SEQ ID NO: 354) | (SEQ ID NO: 357) |
| CDR Sequences (IMGT) | CDR Sequences (IMGT) |
| CDR-H1 GFTFSRYW | CDR-L1 QRVSSSY |
| (SEQ ID NO: 358) | (SEQ ID NO: 361) |
| CDR-H2 IKQDGSEK | CDR-L2 DA |
| (SEQ ID NO: 359) | CDR-L3 QQYGSLPWT |
| CDR-H3 AREGGWFGELAFDY | (SEQ ID NO: 362) |
| (SEQ ID NO: 360) | |
| CDR Sequences (Chothia) | CDR Sequences (Chothia) |
| CDR-H1 GFTFSRY | CDR-L1 RASQRVSSSYLA |
| (SEQ ID NO: 363) | (SEQ ID NO: 366) |
| CDR-H2 KQDGSE | CDR-L2 DASSRAT |
| (SEQ ID NO: 364) | (SEQ ID NO: 367) |
| CDR-H3 EGGWFGELAFDY | CDR-L3 QQYGSLPWT |
| (SEQ ID NO: 365) | (SEQ ID NO: 368) |

### 3. Fc Polypeptides

In some embodiments, the Fc polypeptide is a single chain Fc (scFc), which comprises two Fc chains linked together by a covalent linker, e.g., via an amino acid linker.

An IgG Fc chain (e.g., IgG1 Fc chain) typically contains two constant heavy domains (CH2 and CH3) and a hinge region connected to the CH2 domain.

In some embodiments, the Fc polypeptide comprises one or more IgG1 Fc chains (e.g., human IgG1 Fc chains or a human Fc chains), that is, the Fc polypeptide comprises an Fc chain that has an amino acid sequence that is substantially similar or identical to that of the chains within a wild type IgG1 Fc.

In some embodiments, the Fc polypeptide comprises one or more human IgG1 Fc chains; that is, the Fc polypeptide comprises an Fc chain that is substantially similar or identical to that of the Fc chains within a wild type human IgG1. In some embodiments, the wild type human IgG1 Fc has an amino acid sequence of SEQ ID NO:4.

In certain embodiments, fragment crystallizable (Fc) polypeptides comprise Fc chains that each have one or more mutations relative to a reference Fc chain of the same Ig class. As explained further herein below, the reference Fc chain may be of, e.g., the IgG1 class.

Unless otherwise noted, numbering of residues within an antibody fragment, e.g., an Fc chain, throughout this disclosure is according to the EU numbering.

In some embodiments, the Fc polypeptide comprises one or more human IgG1 Fc chains that is, except for mutations noted herein, the Fc polypeptide comprises an Fc chain that is substantially similar to that of the Fc chains within a wild type human IgG1.

In some embodiments, the Fc polypeptide comprises one or more IgG1 Fc chains (e.g., human IgG1 Fc chains or a human Fc chains), that is, except for having particular residue(s) (which may be different than the residue(s) in the corresponding wild type Fc chains) at certain positions as noted herein, the Fc polypeptide comprises an Fc chain that has an amino acid sequence that is substantially similar to that of the chains within a wild type IgG1 Fc. In some embodiments, the wild type IgG1 Fc is a human IgG1 Fc, in which each Fc chain has an amino acid sequence of SEQ ID NO:4. For example, an Fc polypeptide may comprise an Fc chain with an amino acid sequence that is at least 85%, at least 87.5%, at least 90%, at least 91%, at least 92%, at least 93%, at least 94%, at least 95%, at least 96%, at least 97%, at least 98%, or at least 99% identical to that of an Fc chain within a wild-type IgG1 Fc. In some embodiments, an Fc polypeptide comprises an Fc chain that comprises the particular residue(s) at certain position(s) specifically described for that Fc chain, but has an amino acid sequence that is otherwise 100% identical to a corresponding Fc chain within a wild type Fc chain, e.g., wild type IgG1 Fc chain.

In some embodiments, the Fc polypeptide comprises an Fc chain that has an amino acid sequence that differs by at least one, at least two, at least three, or at least four amino acid residues from the sequence of SEQ ID NO:4. In some embodiments, the Fc polypeptide comprises an Fc chain that has an amino acid sequence that differs by no more than ten, no more than nine, no more than eight, no more than seven, no more than six, no more than five, or no more than four amino acid residues from the sequence of SEQ ID NO:4.

In some embodiments, the Fc chain comprise one or more IgG1, IgG2, IgG3, or IgG4 heavy chain constant regions, e.g., one or more heavy chain constant region having at least 93%, at least 94%, at least 95%, at least 96%, at least 97%, at least 98%, at least 99%, or identical to that of SEQ ID NO:371, 372, 373, or 374.

In some embodiments, the Fc polypeptide comprises an “effector silent” Fc chain that is not capable of binding, or only binds at substantially decreased levels, to one or more Fcγ receptors (e.g., human Fcγ receptors hFcγRI, hFcγRIIa, hFcγRIIb, hFcγRIIIa, hFcγRIIIb). In some embodiments, the Fc polypeptide comprises an Fc chain with a half-life extending mutation or set of mutations.

For example, in some embodiments, the Fc polypeptide comprises an IgG1 Fc with an “LLRAL” mutation: L234A, L235A, G236R, G237A, and A330L, with the amino acid residue at position P329 being proline (same as in the wild type)).

### 4. Linkers

In certain embodiments, linkers are used within fusion polypeptides and/or within single-chain molecules such as scFcs. In some embodiments, the linker is an amino acid linker. For example, a linker as employed herein may comprise from about 1 to about 100 amino acid residues, e.g., about 1 to about 70, about 2 to about 70, about 1 to about 30, or about 2 to about 30 amino acid residues. In some embodiments, the linker comprises at least 2, at least 3, at least 4, at least 5, at least 6, at least 7, at least 8, at least 9, or at least 10 amino acid residues.

In certain embodiments, the linker comprises a glycine-serine sequence, e.g., a (GₙS)ₘ sequence (e.g., GGS, GGGS (SEQ ID NO: 6), and/or GGGGS (SEQ ID NO: 7) sequence) that is present in at least 1, at least 2, at least 3, at least 4, at least 5, at least 6, at least 7, at least 8, at least 9, at least 10, at least 11, at least 12, at least 13, or at least 14 copies within the linker.

### B. Self-Assembled Polypeptide Complexes

In one aspect, provided are self-assembled polypeptide complexes comprising a plurality of fusion polypeptides as disclosed herein. Generally, provided self-assembled polypeptide complexes comprise (a) a plurality of first cytokine-binding fusion polypeptides, each first cytokine-binding fusion polypeptide comprising (1) a first cytokine-binding moiety linked to (2) a nanocage monomer or subunit thereof, and (b) a plurality of Fc fusion polypeptides, each Fc fusion polypeptide comprising (1) an Fc polypeptide linked to (2) a nanocage monomer or subunit thereof. In some embodiments, provided self-assembled complexes further comprise (c) a plurality of second cytokine-binding fusion polypeptides, each second cytokine-binding fusion polypeptide comprising (1) a second cytokine-binding moiety linked to (2) a nanocage monomer or subunit thereof, wherein the second cytokine-binding moiety is distinct from the first cytokine-binding moiety. In certain embodiments, self-assembled polypeptide complexes further comprise a plurality of tumor-binding fusion polypeptides, each tumor-binding fusion polypeptide comprising (1) a tumor-binding moiety linked to (2) a nanocage monomer or subunit thereof.

In some embodiments, the nanocage monomer is a ferritin monomer, and each fusion polypeptide within the self-assembled polypeptide complex comprises a ferritin light chain or a subunit of a ferritin light chain. In these embodiments, the self-assembled polypeptide complex does not comprise any ferritin heavy chains, subunits of ferritin heavy chains, or other ferritin components capable of binding to iron or capable of ferroxidase activity.

In some embodiments, the nanocage monomer or subunit thereof is a ferritin monomer subunit, and (a) each first cytokine-binding fusion polypeptide comprises a ferritin monomer subunit which is C-half-ferritin and each Fc fusion polypeptide comprises a ferritin monomer subunit which is N-half-ferritin; or (b) each first cytokine-binding fusion polypeptide comprises a ferritin monomer subunit which is N-half ferritin and each Fc fusion polypeptide comprises a ferritin monomer subunit which is C-half-ferritin.

In some embodiments in which the self-assembled polypeptide complex further comprises a plurality of second cytokine-binding fusion polypeptides, the nanocage monomer or subunit thereof is a ferritin monomer subunit, and (a) each second cytokine-binding fusion polypeptide comprises a ferritin monomer subunit which is C-half-ferritin and each Fc fusion polypeptide comprises a ferritin monomer subunit which is N-half-ferritin; or (b) each second cytokine-binding fusion polypeptide comprises a ferritin monomer subunit which is N-half ferritin and each Fc fusion polypeptide comprises a ferritin monomer subunit which is C-half-ferritin.

In some embodiments, the self-assembled polypeptide complex comprises between 24 and 48 fusion polypeptides in total. In some embodiments, the self-assembled polypeptide complex comprises 24 fusion polypeptides in total. In some embodiments, the self-assembled polypeptide complex comprises more than 24 fusion polypeptides, e.g., at least 26, at least 28, at least 30, at least 32 fusion polypeptides, at least 34 fusion polypeptides, at least 36 fusion polypeptides, at least 38 fusion polypeptides, at least 40 fusion polypeptides, at least 42 fusion polypeptides, at least 44 fusion polypeptides, at least 46 fusion polypeptides, or at least 48 fusion polypeptides in total. In some embodiments, the self-assembled polypeptide complex comprises about 32 fusion polypeptides.

In some embodiments, the self-assembled polypeptide complex comprises at least 4, at least 5, least 6, at least 7, or at least 8 first cytokine-binding fusion polypeptides.

In some embodiments, the self-assembled polypeptide complex comprises at least 4, at least 5, least 6, at least 7, or at least 8 Fc fusion polypeptides.

In some embodiments, the self-assembled polypeptide complex further comprises at least 4, at least 5, least 6, at least 7, at least 8, at least 9, at least 10, least 11, at least 12, at least 13, at least 14, at least 15, or at least 16 fusion polypeptides distinct from the first cytokine-binding fusion polypeptide and the Fc fusion polypeptide, e.g., a second cytokine-binding fusion polypeptide and/or tumor-binding fusion polypeptide (e.g., a fusion polypeptide comprising a tumor-binding moiety linked to nanocage monomer or a subunit thereof).

In some embodiments, the self-assembled polypeptide complex comprises a ratio of approximately 1:1, 11:13, 3:5, 1:2, 7:17, 1:3, 2:7, 5:19, 1:4, 1:5, 1:6, 1:7, 1:8, 1:12, 1:24 of first cytokine-binding fusion polypeptides to all other fusion polypeptides.

In some embodiments, the self-assembled polypeptide complex comprises a ratio of approximately 1:1, 11:13, 3:5, 1:2, 7:17, 1:3, 2:7, 5:19, 1:4, 1:5, 1:6, 1:7, 1:8, 1:12, 1:24 of first cytokine-binding fusion polypeptides to Fc fusion polypeptides.

In some embodiments, the self-assembled polypeptide complex comprises a ratio of approximately 1:1, 11:13, 3:5, 1:2, 7:17, 1:3, 2:7, 5:19, 1:4, 1:5, 1:6, 1:7, 1:8, 1:12, 1:24 of second cytokine-binding fusion polypeptides to all other fusion polypeptides.

In some embodiments, the self-assembled polypeptide complex comprises a ratio of approximately 1:1, 11:13, 3:5, 1:2, 7:17, 1:3, 2:7, 5:19, 1:4, 1:5, 1:6, 1:7, 1:8, 1:12, 1:24 of second cytokine-binding fusion polypeptides to Fc fusion polypeptides.

### Pharmacokinetic Characteristics

In certain embodiments, when administered to a subject in need thereof, a provided self-assembled polypeptide complex, has one or more pharmacokinetic features similar to that of a reference IgG molecule (e.g., an IgG molecule whose class matches the class of an Fc chain within an Fc polypeptide of a first fusion polypeptide within the self-assembled polypeptide complex).

### Effects

In certain embodiments, disclosed self-assembled polypeptide complexes are capable of sequestering cytokines from the extracellular space and precluding binding to their cognate receptors, thereby abrogating signaling (e.g., inflammatory signaling) mediated by the cytokine(s).

Any of a number of assays known in the art can be used to assess the inhibitory effect of Multabodies disclosed herein on intracellular signaling mediated by cytokines and their corresponding receptors in vitro. For example, in the context of IL-6 signaling, an assay that detects phospho-STAT3 (pSTAT3) may be used to assess IL-6Rα activation. Another assay suitable for testing IL-6 signaling is a cell-based bioassay (e.g., Bio-Glo™ assay) that uses an engineered cell expressing a fluorescent protein (e.g., Firefly luciferase or FLuc) under control of a transcriptional regulator that is responsive to one or more signals induced by activation of the IL-6 receptor by IL-6. In such an assay, the magnitude of fluorescence scales with the intensity of IL-6 signaling and can therefore, be used to assess the impact of Multabodies disclosed herein on IL-6 signaling. As would be appreciated by one of skill in the art, similar assays can be performed using different cytokine systems.

### C. Methods of Treatment

In one aspect, provided are methods that may be useful for treating, ameliorating, or preventing a disease or a condition, generally comprising a step of administering a self-assembled polypeptide complex of the present disclosure (or a composition thereof) to a subject.

In some embodiments, the subject is a mammal, e.g., a human.

Compositions for administration to subjects generally comprise a self-assembled polypeptide complex as disclosed herein. In some embodiments, such compositions further comprise a pharmaceutically acceptable excipient.

Compositions may be formulated for administration for any of a variety of routes of administration, including systemic routes (e.g., oral, inhalation, intranasal, intravenous, intraperitoneal, subcutaneous, or intramuscular administration).

In some embodiments, the step of administering results in improvement in one or more clinical outcomes or metrics in the subject.

In some embodiments, the subject has a cancer, e.g., a solid cancer or a hematological cancer. In some embodiments, the step of administering results in slowing or inhibiting progression of the tumor, e.g., regression of the tumor. In some embodiments, the step of administering results in complete regression of the tumor.

In some embodiments, the subject has an autoimmune disease or disorder.

In some embodiments, the step of administering results in a reduction in one or more symptoms of the autoimmune disease or disorder, e.g., reduction in levels of inflammation, reduction in frequency of inflammatory episodes, reduction in magnitude of one or more symptoms of the autoimmune disease or disorder, etc. In some embodiments, the step of administering results in complete elimination of symptoms of the autoimmune disease or disorder.

### EXAMPLES

### Example 1. Construction and Expression of Anti-IL-6 Multabodies

This Example describes the generation of anti-IL-6 Multabodies (MBs) comprising a combination of fusion proteins comprising a (1) human ferritin light chain or subunit thereof and (2) a single-chain Fab (scFab) or a single chain Fc-dimer (scFc), fused via a linker, such as a (Glyₙ-Ser)ₘ amino acid linker as described herein.

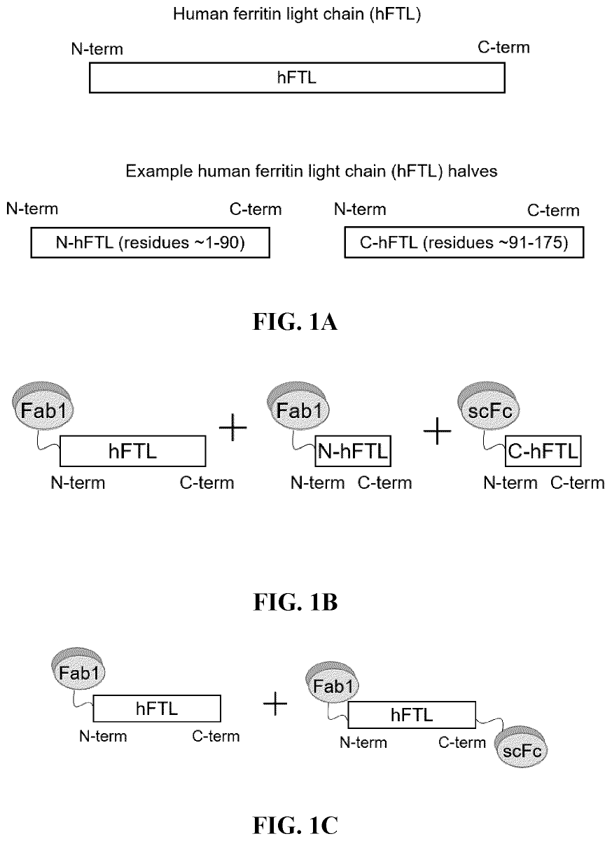

Genes encoding fusion proteins (1) scFab of an anti-IL-6 fused to the N-terminus of full-length ferritin, (2) scFab of an anti-IL-6 fused to the N-terminus of an N-half ferritin, and (3) scFc fused to the N-terminus of a C-half ferritin were prepared, mixed at a molar ratio of 2:1:1 and transiently transfected into ExpiCHO-S cells for the production and formation of the anti-IL-6 MBs. See FIG. 1B.

Three different monospecific Multabodies were made, each using an scFab of a different IL-6 antibody: siltuximab, clazakizumab, or olokizumab.

The scFc-ferritin fusion proteins comprised engineered IgG1 Fc chains which contained the “LLRAL” mutation (L234A, L235A, G236R, G237A, and A330L; with the amino acid residue at position P329 being proline (same as in the wild type)), as shown in Table 1 below. Numbering in Table 4 is according to the EU numbering scheme. This “LLRAL” combination of mutations render the IgG1 Fc “silent” with respect to effector activity, consistent with significantly diminished or no binding to Fcγ receptors (data not shown).

TABLE 4
Residues at certain positions within IgG1 Fc chains
used in Multabodies and corresponding SEQ ID NOs of
scFc-C hFTL constructs
In rows corresponding to sets of engineered Fc chains,
residues are only shown at positions that differ from
the wild type residue.

| | Amino acid position | | | | | | |
|---|---|---|---|---|---|---|---|
| | | | | | | | SEQ ID NO: of |
| | | | | | | | corresponding |
| | | | | | | | scFc-C_hFTL |
| | 234 | 235 | 236 | 237 | 329 | 330 | construct |
| WT | L | L | G | G | P | A | SEQ ID NO: |
| (wild type) | | | | | | | 331 |
| LLRAL | A | A | R | A | | L | SEQ ID NO: |
| | | | | | | | 332 |

### Example 2. Anti-IL-6 Multabodies Bind to IL-6 with High Affinity

The binding kinetics and affinities of the monospecific anti-IL-6 Multabodies produced as described in Example 1 were tested using the Octet® bio-layer interferometry (BLI) system and compared against the IgG antibodies (siltuximab, clazakizumab, and olokizumab) from which the scFabs displayed on the Multabodies were derived. This method measures binding reactions at the surface of a fiber optic biosensor functionalized with an immobilized target moiety (e.g., IL-6).

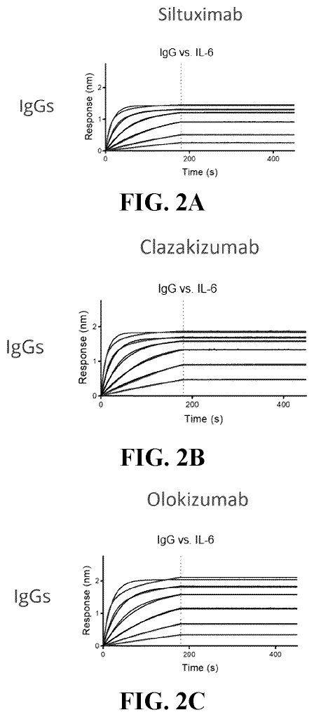

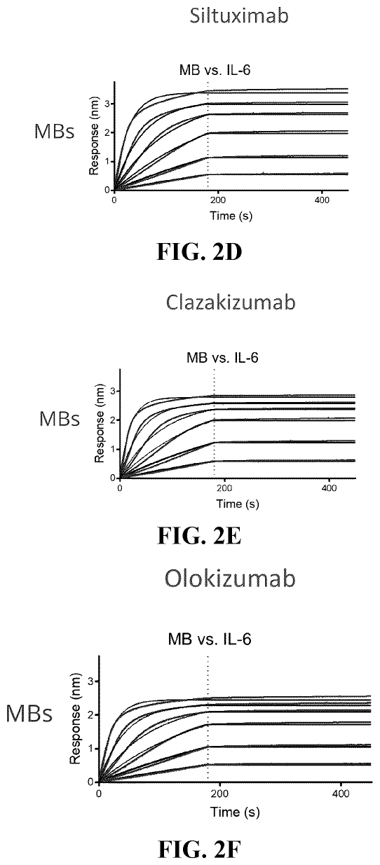

As can be seen from FIGS. 2A and 2D, FIGS. 2B and 2E, and FIGS. 2C and 2F, both anti-IL-6 Multabodies and anti-IL-6 IgGs bound to IL-6 with sub-picomolar binding affinities (K_{D}<1.0×10⁻¹²).

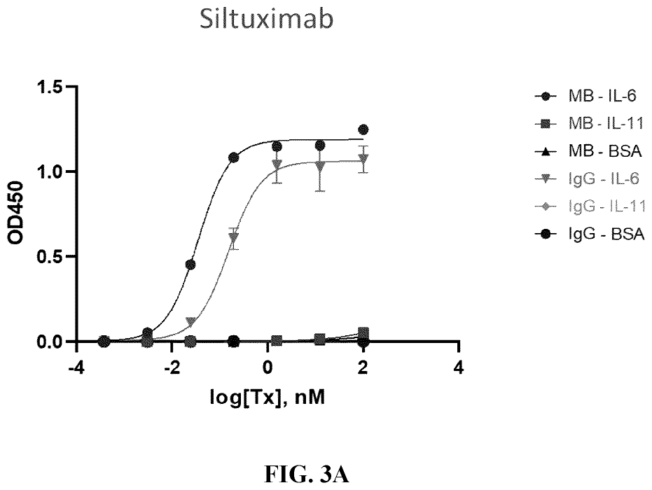

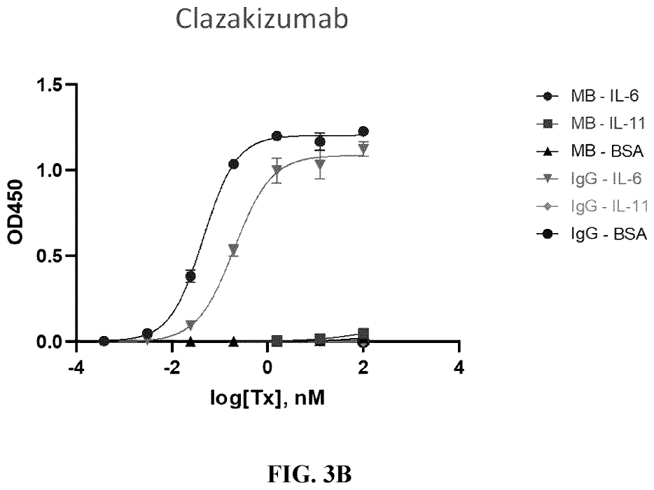

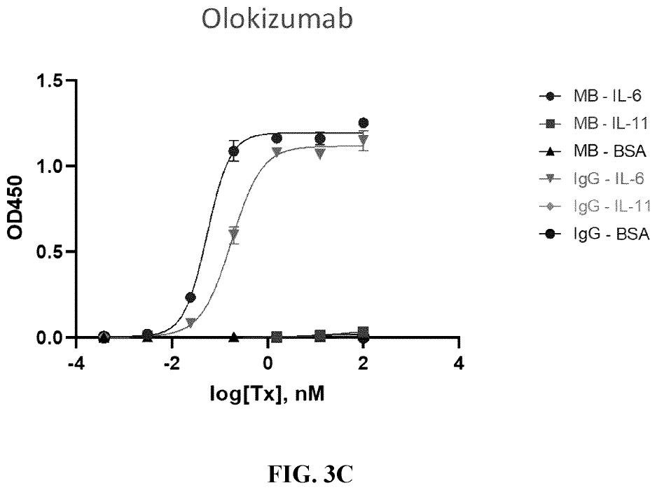

Relative binding and specificity of anti-IL-6 Multabodies were further tested using an ELISA assay and compared against their corresponding IgGs. The results are shown in FIGS. 3A-3C, and the EC₅₀ values are shown in Table 5 below. Anti-IL-6 Multabodies exhibited about 3- to 4-fold enhanced binding compared to corresponding anti-IL6 IgGs. Both anti-IL-6 Multabodies and corresponding anti-IL-6 IgGs exhibited highly selective binding to IL-6, as can be seen from the near-complete absence of binding to surfaces functionalized with IL-11 or bovine serum albumin (BSA).

TABLE 5
EC₅₀ Binding of anti-IL-6 Multabodies and IgGs

EC₅₀ (pM)

| Source Antibody | IgG | Multabody |
|---|---|---|
| Siltuximab | 154.3 | 35.31 |
| Clazakizumab | 176.2 | 54.28 |
| Olokizumab | 200.8 | 45.36 |

### Example 3. Target Binding of Multabodies Determined by Biolayer Interferometry

The binding kinetics and affinity of exemplary anti-IL-6 Multabodies generated as described in Example 1 to IL-6 can be determined by biolayer interferometry (BLI). For example, the binding characteristics of IgG antibodies (from which the scFabs within the IL-6 MBs are derived) can also be determined for comparison.

For example, Ni-NTA biosensors can be coated with His-tagged antigens (e.g., IL-6). Coated biosensors can be dipped into wells containing serial dilutions of the Multabodies (or of the antibody controls) in a buffer such as a phosphate-buffered saline (PBS) buffer optionally comprising Tween 20 and optionally comprising bovine serum albumin (BSA) during an association phase and during a dissociation phase.

Biosensors can be regenerated between experiments, for example, by applying an acidic glycine solution several times and by recharging in NiSO₄.

Target binding can be evaluated based on the maximal association binding response at the end of the association phase, the dissociation rate (k_{off}), and/or the equilibrium dissociation constant (K_{D}) calculated using a 1:1 fitting model. A maximal association binding response of less than 0.1 nm for a concentration of 20 nM Multabody can be classified as “non-binding.”

### Example 4. Binding of Multabodies to Fc Receptors Determined by Biolayer Interferometry

The binding kinetics and affinities of various Multabodies generated as described in Example 1 to various Fc receptors can be determined by biolayer interferometry.

Multabodies tested in this Example contain polypeptides comprising one or more Fc chains. As controls for comparison, Multabodies without Fc-containing polypeptides and/or IgG antibodies of the same class as the Fc chains used in the Multabodies can be used.

Binding to various Fc receptors can be determined, for example, to human Fc receptors, such as: human Fc gamma receptor type I (hFcγRI), hFcγRIIa, hFcγRIIb, hFcγRIIIa, hFcγRIIIb, and human neonatal Fc receptor (hFcRn). To assess the possibility and relevance of testing the Multabodies in animal models, binding to non-human Fc receptors can also be assessed, such as to cynomolgus Fc receptors (e.g., cynomolgus FcγRI (cFcγRI), cFcγRIIa, cFcγRIIb, cFcγRIII, and cFcRn) and/or to mouse Fc receptors (e.g., mouse FcγRI (mFcγRI), mFcγRIIb, mFcγRIII, mFcγRIV, and mFcRn).

Experiments can be performed similarly as described in Example 3, except that His-tagged Fc receptors are coated onto Ni-NTA biosensors and titrated with test Multabodies at various concentrations. To assess the potential of the Multabodies to undergo endosomal recycling, binding to FcRn can be measured at pH 6.0 for association and at pH 7.4 for dissociation.

### Example 5. Anti-IL-6 Multabodies Modulate IL-6 Intracellular Signaling

Binding of IL-6 to its receptor, IL-6Rα, enables its association with the signal-transducing gp130 receptor subunit. Association of gp130 with the IL-6-IL-6Rα complex promotes gp130 dimerization, activating multiple intracellular signaling pathways including the JAK-STAT pathway that involves phosphorylation of STAT3. Thus, phosphorylated STAT3 (pSTAT3) can be used as an indication of IL-6Rα activation.

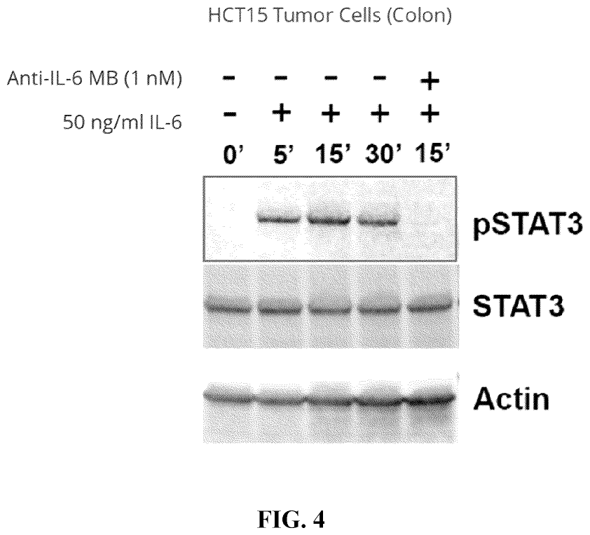

To test whether anti-IL-6 Multabodies of the disclosure can interfere with IL-6 signaling, an in vitro functional assay was performed on HCT-15 colon tumor cells incubated with 50 ng/mL IL-6 in the presence or absence of 1 nM anti-IL-6 Multabody. Cell lysates were prepared, and samples were assessed by western blot using total STAT3 and pSTAT3 antibodies. Actin staining was used as a loading control. As shown in FIG. 4, pSTAT3 was detected in samples from cells incubated with IL-6 alone for as little as five minutes. However, in samples from cells incubated with both IL-6 and an anti-IL-6 Multabody, pSTAT3 was not detected even after 15 minutes.

These results demonstrate that anti-IL-6 Multabodies are potent antagonists of IL-6 signaling in tumor cells.

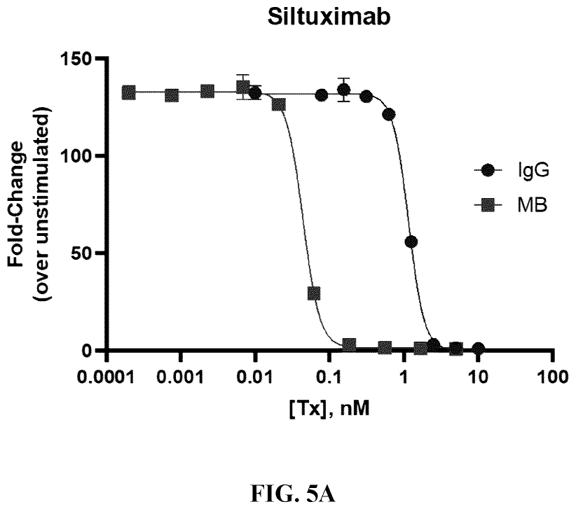

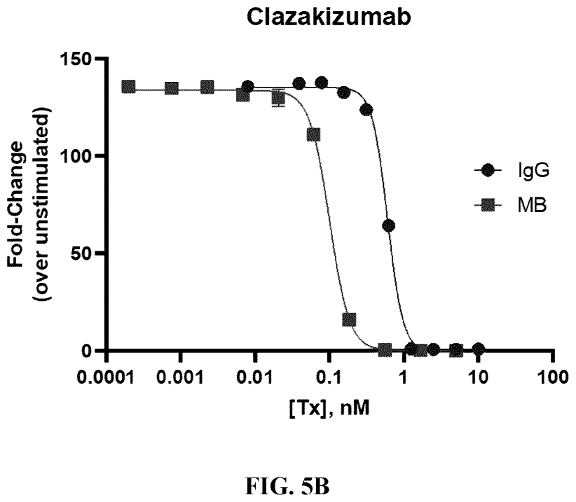

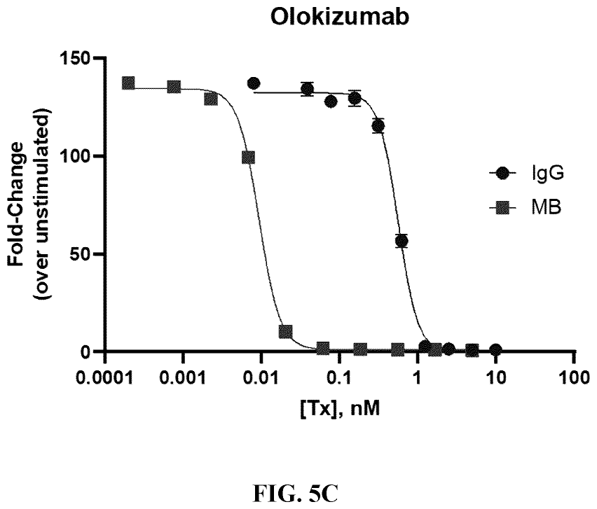

The antagonistic activity of anti-IL-6 Multabodies was further assessed in an IL-6 Bioassay using engineered cells with a luciferase transgene driven by a gene element responsive to IL-6Rα downstream signals. As depicted in FIGS. 5A-5C, anti-IL-6 Multabodies inhibited IL-6 signaling with enhanced potency compared to the corresponding parental anti-IL-6 IgGs. Table 6, below shows the IC50 values calculated from the results of the above experiments.

TABLE 6
Suppression of IL-6 signaling by anti-IL-6 Multabodies and IgGs

| | | | Fold change |
|---|---|---|---|
| | IC₅₀ (pM) | | (IgG vs. |
| Source Antibody | IgG | Multabody | Multabody) |
| Siltuximab | 1154 | 44.01 | 26 |
| Clazakizumab | 604.4 | 100.2 | 6 |
| Olokizumab | 566.2 | 9.4 | 60 |

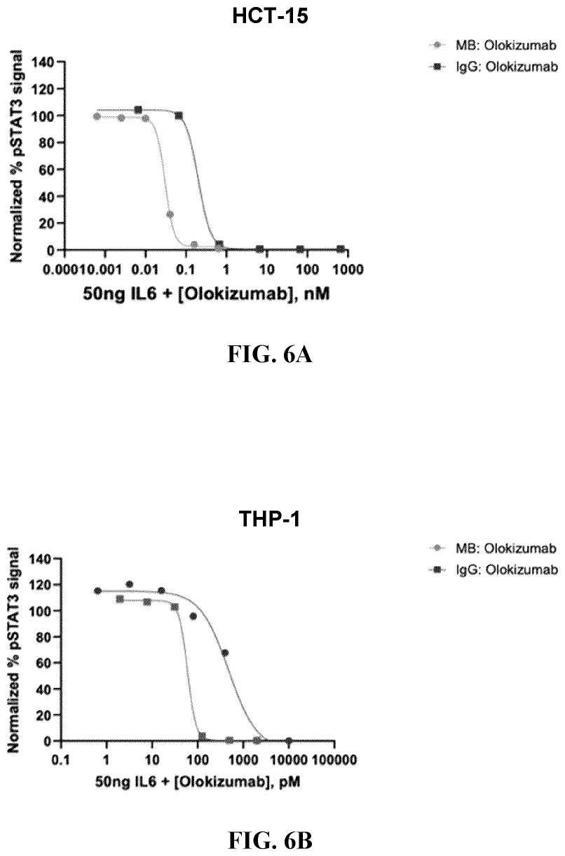

Modulation of IL-6 signaling was also further tested using an assay for IL-6-dependent pSTAT/STAT3 levels. This assay was performed on both HCT-15 and THP-1 cells (a human leukemia monocytic cell line). HCT-15 cells were plated at 2.5×10⁶ cells per 6 cm dish, incubated one day in complete media at 37° C., 5% CO₂, then starved in 3 mL serum free media overnight at 37° C., 5% CO₂ prior to treatment. THP-1 cells were starved overnight at 4.5×10⁶ cells in 3 mL of serum-free media per 6 cm dish at 37° C., 5% CO₂ prior to treatment. MBs or IgGs were pre-incubated with IL-6 (Peprotech) for 10 min., then added to cells to bring the final volume to 4 mL, with the final concentration of IL-6 being 50 ng/mL. Cells were incubated for 15 min. at 37° C., 5% CO₂, and then placed on ice. Cells were pelleted, washed with ice-cold PBS, lysed with 150 μL cold lysis buffer (Meso Scale Discovery). The lysate was centrifuged at 14,000×g for 15 min. and supernatant was collected. Protein concentration was determined by BCA assay kit (Pierce). pSTAT3/STAT3 was determined using phospho-STAT3 and Total STAT3 kits according to the manufacturer's instructions. As shown in FIGS. 6A and 6B, anti-IL-6 Multabodies inhibited IL-6-induced STAT3 signaling with enhanced potency compared to the corresponding parental anti-IL-6 IgGs, consistent with the results from the IL-6 Bioassay.

Table 7 below shows the IC₅₀ values calculated from the results of the above experiments.

TABLE 7
Suppression of IL-6-induced STAT3 signaling
by anti-IL-6 Multabodies and IgGs

| | | |
|---|---|---|
| Source | IC₅₀ (nM) | Fold change |

| Cell line | antibody | IgG | MB | (IgG/MB) |
|---|---|---|---|---|
| HCT-15 | Olokizumab | 0.20 | 0.03 | 7 |
| THP-1 | Olikizumab | 0.48 | 0.06 | 8 |

These results demonstrate that anti-IL-6 Multabodies are potent inhibitors of IL-6 intracellular signaling with enhanced potency compared to that of the parental IgGs.

### Example 6. Pharmacokinetics of Multabodies in a Non-Human Animal Models

The pharmacokinetics (PK) of exemplary anti-IL-6 Multabodies generated as described in Example 1 can be analyzed in one or more non-human animal models, such as mice and/or non-human primates (e.g., cynomolgus monkeys).

Animals receive a single bolus injection of the Multabody or a control molecule (e.g., an IgG) by systemic route of administration (e.g., intravenous). Blood samples are collected at various timepoints, e.g., at around of 3 h, 24 h, 48 h, 72 h, 5 days, 7 days, 14 days, 21 days and 28 days after injection.

Multi-dose studies can also be performed, with the first and second doses spaced apart, e.g., by a number of days or weeks.

Plasma samples may be obtained from blood samples and stored frozen until use.

To measure levels of Multabodies or control molecules in plasma by enzyme-linked immunosorbent assay (ELISA), recombinant IL-6 is coated onto plates and left overnight at 4° C. After washing twice (e.g., with a solution such as phosphate-buffered saline (PBS)-Tween-20, the plate is blocked with a bovine serum albumin (BSA) solution in PBS for 1 hour at room temperature and subsequently washed twice. Plasma samples are diluted in PBS/Tween-20/BSA solution, added to wells, and incubated for 1 h at room temperature on a shaker. After another wash step, bound molecules are detected by incubation with diluted goat polyclonal anti-human Fc-HRP secondary antibody. After a further wash step, a substrate used for detection is added, and the absorbances at 450 nm were read using a microplate reader. A calibration curve is prepared using dilutions of Multabodies in PBS/Tween-20/BSA solution.

Half-lives of Multabodies in animals can be calculated based on these measurements, and the half-lives of Multabodies can be compared to those of IgG molecules.

### Example 7. Therapeutic Efficacy of Anti-IL6 Multabodies in a Xenograft Mouse Model

The therapeutic effect of exemplary Multabodies can be evaluated in a colorectal cancer (CRC) xenograft model.

Mouse models: BALB/c mice and C57BL/6J mice can be used to establish CT26 and MC38 syngeneic mouse models, respectively. Cells can be harvested from cell culture flasks, washed, and resuspended in Matrigel to a concentration of 1×10⁶ cells/100 μl. 100-μl of cell suspension can be injected subcutaneously into a flank of each mouse.

Treatment: After tumors are established, mice can be administered an anti-IL-6 Multabody of the present disclosure or a control (e.g., an IL-6 antibody (IgG), an IgG isotype control, and/or a control Multabody that does not bind to anti-IL-6), for example, by intraperitoneal injection, according to a dosing schedule to be determined by the investigator.

Assessment of therapeutic effect: Tumor growth, survival curves and weight loss can be compared across treatment groups. Tumor length and width are measured weekly using calipers. Reduction in tumor growth and/or improved survival in animals treated with Multabodies (as compared with those treated with control molecules) would suggest improved efficacy of anti-IL-6 Multabodies.

### Example 8. Anti-IL-6 Multabodies Potently Inhibit IL-6 Dependent Cell Proliferation

Anti-IL-6 Multabodies (MBs) generated as described in Example 1 were assessed for ability to inhibit IL-6-dependent cell proliferation.

Cellproliferation assay. Cell proliferation was assessed using the CellTiter-Glo Assay, which determines the number of viable cells in culture by quantitating the amount of ATP present. In this assay, a luminescent signal is proportional to the amount of ATP present, which in turn is directly proportional to the number of viable cells in the culture.

INA-6 cells (which are IL-6 dependent cells) were maintained at 0.5-1.5×10⁶ cells/mL in 80% RPMI 1640+20% FBS+10 ng/mL IL-6 (Peprotech). 2.5×10⁴ cells were plated per well of a 96-well plate in 75 μL 90% RPMI 1640+10% FBS. MBs or IgGs were pre-incubated with IL-6 for 10 min. in 90% RPMI 1640+10% FBS, then added to the cells to bring the final volume to 100 μL, with the final concentration of IL-6 being 17.5 ng/mL. Cells were incubated for three days at 37° C., 5% CO₂. Final viable cell number was determined using CellTiter-Glo 2.0 reagent (Promega) according to the manufacturer's instructions. Proliferation was calculated by fold-change in signal over that of unstimulated cells (no IL-6).

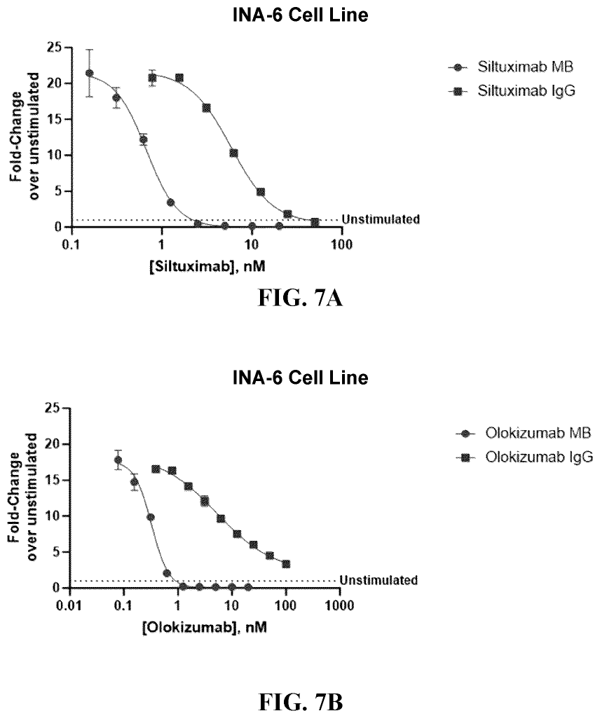

Results. Anti-IL-6 Multabodies potently inhibited cell proliferation with IC₅₀ values several fold lower than the IC₅₀ values for corresponding parental IgGs (FIGS. 7A and 7B and Table 8).

TABLE 8
Inhibition of cell proliferation by anti-IL-6 Multabodies and IgGs

| | IC₅₀ (nM) | | |
|---|---|---|---|
| Source antibody | MB | IgG | Fold change |
| Siltuximab | 0.67 | 5.88 | 9 |
| Olokizumab | 0.33 | 5.29 | 16 |

### Example 9. Construction, Expression, and Analysis of anti-IL-11 Multabodies

This Example describes the generation and analysis of anti-IL-11 Multabodies (MBs).

### Construction and Expression of Anti-IL-11 Multabodies

Anti-IL-11 Multabodies were generated similarly as described in Example 1, except that the scFabs within certain fusion proteins were the scFabs derived with variable regions from an IL-11 antibody instead of an IL-6 antibody.

### Assessment of Binding of Anti-IL-11 Multabodies to IL-11

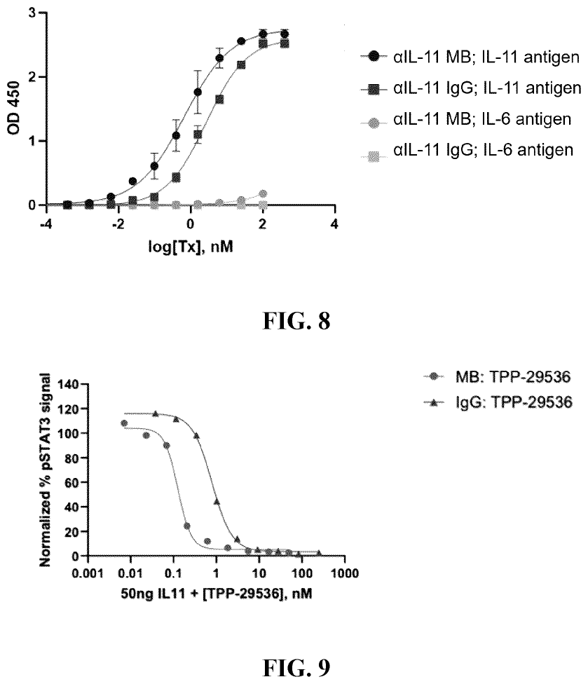

Binding of anti-IL-11 Multabodies to IL-11 was assessed using an enzyme-linked immunosorbent assay (ELISA). As shown in FIG. 8 and Table 9, anti-IL-11 Multabodies (MB) bound IL-11 about 3-4 fold better than did the parental IgG control in the same assay. Both anti-IL-11 MBs and anti-IL-11 IgGs exhibited minimal binding to IL-6.

TABLE 9
Binding to IL-11 by anti-IL-11 Multabodies and IgGs

EC₅₀ (pM)

| Source antibody | IgG | MB |
|---|---|---|
| TPP-29536 | 2710 | 658.6 |

### Assessment of Modulation of IL-11-Induced STAT3 Signaling

Modulation of IL-11 signaling was also tested using an assay for IL-11-dependent pSTAT/STAT3 levels. Panc-1 cells (pancreatic cancer cells) were plated at 2.5×10⁶ cells in complete media in a 6 cm dish and incubated one day at 37° C., 5% CO₂ then starved overnight in 3 mL of serum-free media prior to treatment. MBs or IgGs were pre-incubated with IL-11 (AcroBiosystems) for 10 min., then added to cells to bring the final volume to 4 mL, with the final concentration of IL-11 being 50 ng/mL. Cells were incubated for 15 min. at 37° C., 5% CO₂, and then placed on ice. Cells were washed with ice-cold PBS and lysed with 175 μL cold lysis buffer. The lysate was centrifuged at 14,000×g for 15 min. and supernatant was collected. Protein concentration was determined by BCA assay kit (Pierce). pSTAT3/STAT3 was determined using phospho-STAT3 and total STAT3 kits (Meso Scale Discovery) according to the manufacturer's instructions.

As shown in FIG. 9 and Table 10, anti-IL-11 Multabodies demonstrated superior potency in inhibiting pSTAT3 signaling compared to that demonstrated by the parental IgG control.

TABLE 10
Inhibition of pSTAT3 signaling by
anti-IL-11 Multabodies and IgGs

| | IC₅₀ (nM) | | |
|---|---|---|---|
| Source antibody | IgG | MB | Fold change |
| TPP-29536 | 0.79 | 0.13 | 6 |

Thus, these experiments demonstrate successful generation of anti-IL-11 Multabodies that bind specifically to IL-11 and inhibit pSTAT3 signaling better than the parental anti-IL-11 IgGs do.

### Example 10. Construction, Expression, and Analysis of Additional Anti-IL-11 Multabodies

Additional anti-IL-11 Multabodies can be generated as described in Example 9, using, e.g., Fabs from other IL-11 antibodies, examples of which are included in Tables 2A-2H.

### Example 11. Pharmacokinetic Analysis Anti-IL-11 Multabodies

The pharmacokinetics (PK) of exemplary anti-IL-11 Multabodies generated as described in Examples 9 and 10 can be analyzed in one or more non-human animal models, such as mice and/or non-human primates (e.g., cynomolgus monkeys), similarly as described in Example 6.

### Example 12. Therapeutic Efficacy of Anti-IL-11 Multabodies

The therapeutic effect of exemplary anti-IL-11 Multabodies can be evaluated in a mouse cancer xenograft model, e.g., a colorectal cancer (CRC) xenograft model, similarly as described in Example 7.

### SEQUENCE LISTING

Underlining within fusion sequences indicate linker sequences.

Bolding within fusion sequences indicate ferritin or ferritin subunit sequences.

Within variable region sequences, underlining and bolding together indicate complementary determining regions sequences.

Boxed and bolded residues indicate residues that are mutated relative to a reference molecule, e.g. relative to an IgG1 Fc.

```
hFTL
                                                                                                    SEQ ID NO: 1
MSSQIRQNYSTDVEAAVNSLVNLYLQASYTYLSLGFYFDRDDVALEGVSHFFRELAEEKREG
YERLLKMQNQRGGRALFQDIKKPAEDEWGKTPDAMKAAMALEKKLNQALLDLHALGSARTDP
HLCDFLETHFLDEEVKLIKKMGDHLTNLHRLGGPEAGLGEYLFERLTLRHD
N_hFTL
                                                                                                    SEQ ID NO: 2
SSQIRQNYSTDVEAAVNSLVNLYLQASYTYLSLGFYFDRDDVALEGVSHFFRELAEEKREGY
ERLLKMQNQRGGRALFQDIKKPAEDEW
C_hFTL
                                                                                                    SEQ ID NO: 3
GKTPDAMKAAMALEKKLNQALLDLHALGSARTDPHLCDFLETHFLDEEVKLIKKMGDHLTNL
HRLGGPEAGLGEYLFERLTLRHD
IgG1_Fc
                                                                                                    SEQ ID NO: 4
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK
IgG1_scFc
                                                                                                    SEQ ID NO: 5
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGGGGGSGGGGSGGGGSGGGGSGGGGSGGGGSDKTHTCPPCPAPEL
LGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQG
NVFSCSVMHEALHNHYTQKSLSLSPGK
Example GS linker
                                                                                                    SEQ ID NO: 6
GGGS
Example GS linker
                                                                                                    SEQ ID NO: 7
GGGGS
SEQ ID NOs: 8-368 as shown in Tables 1A-4D.
scFc-C_hFTL IgGl WT
                                                                                                SEQ ID NO: 369
DKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGKGGGGSGGGGSGGGGSGGGGSG
GGGSGGGGSGGGGSGGGGGGGGSGGGGSGGGGSGGGGSGGGGSGGGGSGGGGSDKPCPAPEL
LGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGGGGSGGGGSGGGKTPDAMK
AAMALEKKLNQALLDLHALGSARTDPHLCDFLETHFLDEEVKLIKKMGDHLTNLHRLGGPEA
GLGEYLFERLTLRHD
scFc-C_hFTL IgG1 LLRAL
                                                                                                SEQ ID NO: 370

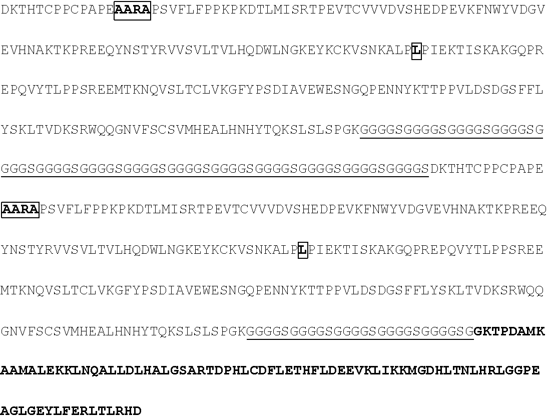

human IgG4 heavy chain constant region sequence
                                                                                                SEQ ID NO: 371
ASTKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPSCPAPEFLGGPSVFLFP
PKPKDTLMISRTPEVTCVVVDVSQEDPEVQFNWYVDGVEVHNAKTKPREEQFNSTYRVVSVL
TVLHQDWLNGKEYKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQEGNVFSCSVMHE
ALHNHYTQKSLSLSLGK
human IgG1 heavy chain constant region sequence
                                                                                                SEQ ID NO: 372
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
human IgG2 heavy chain constant region sequence
                                                                                                SEQ ID NO: 373
ASTKGPSVFPLAPCSRSTSESTAALGCLVKDYFPEPVTVSWNSGALTSGVHTFPAVLQSSGL
YSLSSVVTVPSSNFGTQTYTCNVDHKPSNTKVDKTVERKCCVECPPCPAPPVAGPSVFLFPP
KPKDTLMISRTPEVTCVVVDVSHEDPEVQFNWYVDGVEVHNAKTKPREEQFENSTFRVVSVL
TVVHQDWLNGKEYKCKVSNKGLPAPIEKTISKTKGQPREPQVYTLPPSREEMTKNQVSLTCL
VKGFYPSDISVEWESNGQPENNYKTTPPMLDSDGSFFLYSKLTVDKSRWQQGNVFSCSVMHE
ALHNHYTQKSLSLSPGK
human IgG3 heavy chain constant region sequence
                                                                                                SEQ ID NO: 374
ASTKGPSVFPLAPCSRSTSGGTAALGCLVKDYFPEPVTVSWNSGALTSGVHTFPAVLQSSGL
YSLSSVVTVPSSSLGTQTYTCNVNHKPSNTKVDKRVELKTPLGDTTHTCPRCPEPKSCDTPP
PCPRCPEPKSCDTPPPCPRCPEPKSCDTPPPCPRCPAPELLGGPSVFLFPPKPKDTLMISRT
PEVTCVVVDVSHEDPEVQFKWYVDGVEVHNAKTKPREEQYNSTFRVVSVLTVLHQDWLNGKE
YKCKVSNKALPAPIEKTISKTKGQPREPQVYTLPPSREEMTKNQVSLTCLVKGFYPSDIAVE
WESSGQPENNYNTTPPMLDSDGSFFLYSKLTVDKSRWQQGNIFSCSVMHEALHNRFTQKSLS
LSPGK
human Igκ light chain constant region sequence
                                                                                                SEQ ID NO: 375
RTVAAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
human Igλ1 light chain constant region sequence
                                                                                                SEQ ID NO: 376
GQPKANPTVTLFPPSSEELQANKATLVCLISDFYPGAVTVAWKADGSPVKAGVETTKPSKQS
NNKYAASSYLSLTPEQWKSHRSYSCQVTHEGSTVEKTVAPTECS
human Igλ2 light chain constant region sequence
                                                                                                SEQ ID NO: 377
GQPKAAPSVTLFPPSSEELQANKATLVCLISDFYPGAVTVAWKADSSPVKAGVETTTPSKQS
NNKYAASSYLSLTPEQWKSHRSYSCQVTHEGSTVEKTVAPTECS
human Igλ3 light chain constant region sequence
                                                                                                SEQ ID NO: 378
GQPKAAPSVTLFPPSSEELQANKATLVCLISDFYPGAVTVAWKADSSPVKAGVETTTPSKQS
NNKYAASSYLSLTPEQWKSHKSYSCQVTHEGSTVEKTVAPTECS
human Igλ6 light chain constant region sequence
                                                                                                SEQ ID NO: 379
GQPKAAPSVTLFPPSSEELQANKATLVCLISDFYPGAVKVAWKADGSPVNTGVETTTPSKQS
NNKYAASSYLSLTPEQWKSHRSYSCQVTHEGSTVEKTVAPAECS
human Igλ7 light chain constant region sequence
                                                                                                SEQ ID NO: 380
GQPKAAPSVTLFPPSSEELQANKATLVCLVSDFNPGAVTVAWKADGSPVKVGVETTKPSKQS
NNKYAASSYLSLTPEQWKSHRSYSCRVTHEGSTVEKTVAPAECS
```

### EQUIVALENTS/OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

1. A fusion polypeptide comprising: (1) a cytokine-binding moiety and (2) a nanocage monomer or subunit thereof.

2. The fusion polypeptide of claim 1, wherein the cytokine-binding moiety comprises an antibody or antigen-binding fragment thereof.

3. The fusion polypeptide of claim 2, wherein the antibody or antigen-binding fragment thereof comprises an immunoglobulin heavy chain variable region (V_{H}) and an immunoglobulin light chain variable region (V_{L} or V_{K}).

4. The fusion polypeptide of claim 3, wherein the antibody or antigen-binding fragment thereof comprises a Fab fragment.

5. The fusion polypeptide of claim 4, wherein the Fab fragment is a single-chain Fab fragment (scFab).

6. The fusion polypeptide of any one of claims 1-5, wherein the cytokine-binding moiety is capable of binding to interleukin-6 (IL-6) or interleukin-11 (IL-11).

7. The fusion polypeptide of claim 6, wherein the cytokine-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of an IL-6 or IL-11 antibody.

8. The fusion polypeptide of claim 7, wherein the CDRs of the V_{H} and the V_{L} or V_{K} have sequences which are identical to those of the heavy and light chain CDRs of an IL-6 or IL-11 antibody, except for one or two amino acid substitutions total across all six CDRs.

9. The fusion polypeptide of claim 8, wherein the CDRs of the V_{H} and the V_{L} or V_{K} are identical to those of the heavy and light chain CDRs of an IL-6 or IL-11 antibody.

10. The fusion polypeptide of claim 9, wherein the cytokine-binding moiety comprises an antibody fragment comprising heavy and light chain variable regions with sequences that have at least 85% identity to those of the heavy and light chain variable regions of an IL-6 or IL-11 antibody.

11. The fusion polypeptide of any one of claims 6-10, wherein the cytokine-binding moiety is capable of binding to an epitope of interleukin-6 (IL-6), which epitope is involved in binding of IL-6 to IL-6 receptor (IL-6Rα).

12. The fusion polypeptide of any one of claims 6-10, wherein the cytokine-binding moiety is capable of binding to an epitope of IL-6 that is involved in binding of IL-6 to glycoprotein 130 (gp130).

13. The fusion polypeptide of any one of claims 1-12, wherein the nanocage monomer is a ferritin monomer or a subunit thereof.

14. The fusion polypeptide of claim 13, wherein the ferritin monomer is a human ferritin monomer.

15. The fusion polypeptide of claim 13 or 14, wherein the ferritin monomer is a ferritin light chain.

16. The fusion polypeptide of any one of claims 1-15, wherein the cytokine-binding moiety is linked via an amino acid linker to the nanocage monomer or subunit thereof.

17. The fusion polypeptide of any one of claims 1-16, wherein the cytokine-binding moiety is linked via the N-terminus of the nanocage monomer or subunit thereof.

18. A self-assembled polypeptide complex comprising:
(a) a plurality of first cytokine-binding fusion polypeptides, each first cytokine-binding fusion polypeptide being a fusion polypeptide of any one of claims 1-15; and
(b) a plurality of Fc fusion polypeptides, each Fc fusion polypeptide comprising (1) an Fc polypeptide and (2) a nanocage monomer or subunit thereof.

19. The self-assembled polypeptide complex of claim 18, further comprising
(c) a plurality of second cytokine-binding fusion polypeptides, each second cytokine-binding fusion polypeptide being a fusion polypeptide of any one of claims 1-15,
wherein the first and second cytokine-binding fusion polypeptides comprise first and second cytokine-binding moieties, respectively, which are capable of binding to different epitopes.

20. The self-assembled polypeptide complex of claim 19, wherein the first and second cytokine-binding moieties are capable of binding to different cytokines.

21. The self-assembled polypeptide complex of claim 20, wherein the first cytokine-binding moiety is capable of binding to IL-6 and the second cytokine-binding moiety is capable of binding to IL-11.

22. The self-assembled polypeptide complex of any one of claims 18-21, further comprising
(d) a plurality of tumor-binding fusion polypeptides, each tumor-binding fusion polypeptide comprising (1) tumor-binding moiety and (2) a nanocage monomer or subunit thereof.

23. The self-assembled polypeptide complex of any one of claims 18-22, wherein, within each
Fc fusion polypeptide, the Fc polypeptide is linked via an amino acid linker to the nanocage monomer or subunit thereof.

24. The self-assembled polypeptide complex of claim 23, wherein the Fc polypeptide is linked via the N-terminus of the nanocage monomer or subunit thereof.

25. The self-assembled polypeptide complex of claim 24, wherein the Fc polypeptide is linked via the C-terminus of the nanocage monomer or subunit thereof.

26. The self-assembled polypeptide complex of any one of claims 22-25, wherein, within
each tumor-binding fusion polypeptide, the tumor-binding moiety is linked via an amino acid linker to the nanocage monomer or subunit thereof.

27. The self-assembled polypeptide complex of claim 26, wherein the tumor-binding moiety is linked via the N-terminus of the nanocage monomer or subunit thereof.

28. The self-assembled polypeptide complex of any one of claims 22-27, wherein the tumor-binding moiety comprises an antibody or antigen-binding fragment thereof.

29. The self-assembled polypeptide complex of claim 28, wherein the antibody or antigen-binding fragment thereof comprises an immunoglobulin heavy chain variable region (V_{H}) and an immunoglobulin light chain variable region (V_{L} or V_{K}).

30. The self-assembled polypeptide complex of claim 29, wherein the antibody or antigen-binding fragment thereof comprises a Fab fragment.

31. The self-assembled polypeptide complex of claim 30, wherein the Fab fragment is a single-chain Fab fragment (scFab).

32. The self-assembled polypeptide complex of any one of claims 28-31, wherein the tumor-binding moiety is a PD-L1-binding moiety.

33. The self-assembled polypeptide complex of claim 32, wherein the tumor-binding moiety comprises an antibody fragment comprising a heavy chain variable region (V_{H}) and light chain variable region (V_{L} or V_{K}),
wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a PD-L1 antibody.

34. The self-assembled polypeptide complex of claim 33, wherein the CDRs of the V_{H} and the V_{L} or V_{K} have sequences which are identical to those of the heavy and light chain CDRs of a PD-L1 antibody, except for one or two amino acid substitutions total across all six CDRs.

35. The self-assembled polypeptide complex of claim 34, wherein the CDRs of the V_{H} and the V_{L} or V_{K} are identical to those of the heavy and light chain CDRs of a PD-L1 antibody.

36. The self-assembled polypeptide complex of claim 32, wherein the tumor-binding moiety comprises an antibody fragment comprising heavy and light chain variable regions with sequences that have at least 85% identity to those of the heavy and light chain variable regions of a PD-L1 antibody.

37. The self-assembled polypeptide complex of any one of claims 18-36, wherein the nanocage monomers within each Fc fusion polypeptide, each cytokine-binding fusion polypeptide, and/or each tumor-binding fusion polypeptide are each a ferritin monomer or a subunit thereof.

38. The self-assembled polypeptide complex of claim 37, wherein the ferritin monomer is a human ferritin monomer.

39. The self-assembled polypeptide complex of claim 37 or 38, wherein the ferritin monomer is a ferritin light chain.

40. The self-assembled polypeptide complex of claim 39, which does not comprise any ferritin heavy chains or subunits of ferritin heavy chains.

41. The self-assembled polypeptide complex of any one of claims 37-40, which does not comprise any iron-binding moieties.

42. A pharmaceutical composition comprising a self-assembled polypeptide complex of any one of claims 18-41 and a pharmaceutically acceptable excipient.

43. Use of the self-assembled polypeptide complex of any one of claims 18-41 or the pharmaceutical composition of claim 42 to treat, ameliorate, or prevent a disease or condition in a subject.

44. The use of claim 43, wherein the subject is a mammal.

45. The use of claim 44, wherein the subject is human.

46. The use of any one of claims 43-45, wherein the disease or condition is cancer.

47. The use of claim 46, wherein the cancer is a solid cancer.

48. The use of claim 46, wherein the cancer is a hematological cancer.

49. The use of any one of claims 43-45, wherein the disease or condition is an autoimmune disease or condition.

50. A method of treating, ameliorating, or preventing a disease or condition, the method comprising administering to a subject the self-assembled polypeptide complex of any of claims 18-41 or the pharmaceutical composition of claim 42.

51. The method of claim 50, wherein the subject is a mammal.

52. The method of claim 51, wherein the subject is human.

53. The method of any one of claims 50-52, wherein the disease or condition is cancer.

54. The method of claim 53, wherein the cancer is a solid cancer.

55. The method of claim 53, wherein the cancer is a hematological cancer.

56. The method of any one of claims 50-52, wherein the disease or condition is an autoimmune disease or condition.


SEQUENCE LISTING (380 sequences)

SEQ ID NO: 1 - protein, 175 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 2 - protein, 89 aa, Homo sapiens
SSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 3 - protein, 85 aa, Homo sapiens
GKTPDAMKAAMALEKKLNQALLDLHALGSARTDPHLCDFLETHFLDEEVKLIKKMGDHLTNLHRLGGPEAGLGEYLFERLTLRHD

SEQ ID NO: 4 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 524 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 4 aa, synthetic construct
GGGS

SEQ ID NO: 7 - protein, 5 aa, synthetic construct
GGGGS

SEQ ID NO: 8 - protein, 120 aa, synthetic construct
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 9 - protein, 111 aa, synthetic construct
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKR

SEQ ID NO: 10 - protein, 5 aa, synthetic construct
NYYVT

SEQ ID NO: 11 - protein, 16 aa, synthetic construct
IIYGSDETAYATSAIG

SEQ ID NO: 12 - protein, 12 aa, synthetic construct
DDSSDWDAKFNL

SEQ ID NO: 13 - protein, 11 aa, synthetic construct
QASQSINNELS

SEQ ID NO: 14 - protein, 7 aa, synthetic construct
RASTLAS

SEQ ID NO: 15 - protein, 12 aa, synthetic construct
QQGYSLRNIDNA

SEQ ID NO: 16 - protein, 8 aa, synthetic construct
GFSLSNYY

SEQ ID NO: 17 - protein, 7 aa, synthetic construct
IYGSDET

SEQ ID NO: 18 - protein, 14 aa, synthetic construct
ARDDSSDWDAKFNL

SEQ ID NO: 19 - protein, 6 aa, synthetic construct
QSINNE

SEQ ID NO: 20 - protein, 12 aa, synthetic construct
QQGYSLRNIDNA

SEQ ID NO: 21 - protein, 7 aa, synthetic construct
GFSLSNY

SEQ ID NO: 22 - protein, 5 aa, synthetic construct
YGSDE

SEQ ID NO: 23 - protein, 12 aa, synthetic construct
DDSSDWDAKFNL

SEQ ID NO: 24 - protein, 11 aa, synthetic construct
QASQSINNELS

SEQ ID NO: 25 - protein, 7 aa, synthetic construct
RASTLAS

SEQ ID NO: 26 - protein, 12 aa, synthetic construct
QQGYSLRNIDNA

SEQ ID NO: 27 - protein, 122 aa, synthetic construct
QVQLVQSGAEVKKPGSSVKVSCKASGYALSNYLIEWVRQAPGQGLEWMGVITPGSGTINYAQKFQGRVTITADESTSTAYMELSSLRSEDTAVYYCARSRWDPLYYYALEYWGQGTTVTVSS

SEQ ID NO: 28 - protein, 112 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIKR

SEQ ID NO: 29 - protein, 5 aa, synthetic construct
NYLIE

SEQ ID NO: 30 - protein, 17 aa, synthetic construct
VITPGSGTINYAQKFQG

SEQ ID NO: 31 - protein, 13 aa, synthetic construct
SRWDPLYYYALEY

SEQ ID NO: 32 - protein, 15 aa, synthetic construct
RASESVDNYGIPFMN

SEQ ID NO: 33 - protein, 7 aa, synthetic construct
AASNRGS

SEQ ID NO: 34 - protein, 9 aa, synthetic construct
QQSEEVPLT

SEQ ID NO: 35 - protein, 8 aa, synthetic construct
GYALSNYL

SEQ ID NO: 36 - protein, 8 aa, synthetic construct
ITPGSGTI

SEQ ID NO: 37 - protein, 15 aa, synthetic construct
ARSRWDPLYYYALEY

SEQ ID NO: 38 - protein, 10 aa, synthetic construct
ESVDNYGIPF

SEQ ID NO: 39 - protein, 9 aa, synthetic construct
QQSEEVPLT

SEQ ID NO: 40 - protein, 7 aa, synthetic construct
GYALSNY

SEQ ID NO: 41 - protein, 6 aa, synthetic construct
TPGSGT

SEQ ID NO: 42 - protein, 13 aa, synthetic construct
SRWDPLYYYALEY

SEQ ID NO: 43 - protein, 15 aa, synthetic construct
RASESVDNYGIPFMN

SEQ ID NO: 44 - protein, 7 aa, synthetic construct
AASNRGS

SEQ ID NO: 45 - protein, 9 aa, synthetic construct
QQSEEVPLT

SEQ ID NO: 46 - protein, 122 aa, synthetic construct
QVQLVQSGAEVKKPGSSVKVSCKASGYVLPNYLIEWVRQAPGQGLEWMGVTTPGGGTINYAQKFQGRVTITADESTSTAYMELSSLRSEDTAVYYCARSRWDPLYYYALEYWGQGTTVTVSS

SEQ ID NO: 47 - protein, 112 aa, synthetic construct; identical to SEQ ID NO: 28

SEQ ID NO: 48 - protein, 5 aa, synthetic construct
NYLIE

SEQ ID NO: 49 - protein, 17 aa, synthetic construct
VTTPGGGTINYAQKFQG

SEQ ID NO: 50 - protein, 13 aa, synthetic construct
SRWDPLYYYALEY

SEQ ID NO: 51 - protein, 15 aa, synthetic construct
RASESVDNYGIPFMN

SEQ ID NO: 52 - protein, 7 aa, synthetic construct
AASNRGS

SEQ ID NO: 53 - protein, 9 aa, synthetic construct
QQSEEVPLT

SEQ ID NO: 54 - protein, 8 aa, synthetic construct
GYVLPNYL

SEQ ID NO: 55 - protein, 8 aa, synthetic construct
TTPGGGTI

SEQ ID NO: 56 - protein, 15 aa, synthetic construct
ARSRWDPLYYYALEY

SEQ ID NO: 57 - protein, 10 aa, synthetic construct
ESVDNYGIPF

SEQ ID NO: 58 - protein, 9 aa, synthetic construct
QQSEEVPLT

SEQ ID NO: 59 - protein, 7 aa, synthetic construct
GYVLPNY

SEQ ID NO: 60 - protein, 6 aa, synthetic construct
TPGGGT

SEQ ID NO: 61 - protein, 13 aa, synthetic construct
SRWDPLYYYALEY

SEQ ID NO: 62 - protein, 15 aa, synthetic construct
RASESVDNYGIPFMN

SEQ ID NO: 63 - protein, 7 aa, synthetic construct
AASNRGS

SEQ ID NO: 64 - protein, 9 aa, synthetic construct
QQSEEVPLT

SEQ ID NO: 65 - protein, 122 aa, synthetic construct
QVTLKESGPGILKPSQTLSLTCSFSGFSLSTSGMGVGWIRQPSGKGLEWLAHIWWDDDKYYNPSLKSQLTISKDTSRNQVFLKITSVDTADTATYYCARSYDDYLYYALDYWGQGTSVTVSS

SEQ ID NO: 66 - protein, 108 aa, synthetic construct
DIVMTQSPATLSVTPGDRVSLSCRASQSISDYLHWYQQKSHESPRLLIKSVSQSISGIPSRFSGSGSGSDFTLSINSVEPEDVGVYYCQNGHSFPLTFGAGTKLELKR

SEQ ID NO: 67 - protein, 7 aa, synthetic construct
TSGMGVG

SEQ ID NO: 68 - protein, 16 aa, synthetic construct
HIWWDDDKYYNPSLKS

SEQ ID NO: 69 - protein, 12 aa, synthetic construct
SYDDYLYYALDY

SEQ ID NO: 70 - protein, 11 aa, synthetic construct
RASQSISDYLH

SEQ ID NO: 71 - protein, 7 aa, synthetic construct
SVSQSIS

SEQ ID NO: 72 - protein, 9 aa, synthetic construct
QNGHSFPLT

SEQ ID NO: 73 - protein, 10 aa, synthetic construct
GFSLSTSGMG

SEQ ID NO: 74 - protein, 7 aa, synthetic construct
IWWDDDK

SEQ ID NO: 75 - protein, 14 aa, synthetic construct
ARSYDDYLYYALDY

SEQ ID NO: 76 - protein, 6 aa, synthetic construct
QSISDY

SEQ ID NO: 77 - protein, 9 aa, synthetic construct
QNGHSFPLT

SEQ ID NO: 78 - protein, 9 aa, synthetic construct
GFSLSTSGM

SEQ ID NO: 79 - protein, 5 aa, synthetic construct
WWDDD

SEQ ID NO: 80 - protein, 12 aa, synthetic construct
SYDDYLYYALDY

SEQ ID NO: 81 - protein, 11 aa, synthetic construct
RASQSISDYLH

SEQ ID NO: 82 - protein, 7 aa, synthetic construct
SVSQSIS

SEQ ID NO: 83 - protein, 9 aa, synthetic construct
QNGHSFPLT

SEQ ID NO: 84 - protein, 120 aa, synthetic construct
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPWIDLWGRGTLVTVSS

SEQ ID NO: 85 - protein, 107 aa, synthetic construct
DIQMTQSPSTLSASVGDRVTITCRASQGISSWLAWYQQKPGKAPKVLIYKASTLESGVPSRFSGSGSGTEFTLTISSLQPDDFATYYCQQSWLGGSFGQGTKLEIKR

SEQ ID NO: 86 - protein, 5 aa, synthetic construct
SNYMI

SEQ ID NO: 87 - protein, 17 aa, unidentified
DLYYYAGDTYYADSVKG

SEQ ID NO: 88 - protein, 11 aa, synthetic construct
WADDHPPWIDL

SEQ ID NO: 89 - protein, 11 aa, synthetic construct
RASQGISSWLA

SEQ ID NO: 90 - protein, 7 aa, synthetic construct
KASTLES

SEQ ID NO: 91 - protein, 8 aa, synthetic construct
QQSWLGGS

SEQ ID NO: 92 - protein, 8 aa, synthetic construct
GFTISSNY

SEQ ID NO: 93 - protein, 8 aa, synthetic construct
LYYYAGDT

SEQ ID NO: 94 - protein, 13 aa, synthetic construct
ARWADDHPPWIDL

SEQ ID NO: 95 - protein, 6 aa, synthetic construct
QGISSW

SEQ ID NO: 96 - protein, 8 aa, synthetic construct
QQSWLGGS

SEQ ID NO: 97 - protein, 7 aa, synthetic construct
GFTISSN

SEQ ID NO: 98 - protein, 6 aa, synthetic construct
YYYAGD

SEQ ID NO: 99 - protein, 11 aa, synthetic construct
WADDHPPWIDL

SEQ ID NO: 100 - protein, 11 aa, synthetic construct
RASQGISSWLA

SEQ ID NO: 101 - protein, 7 aa, synthetic construct
KASTLES

SEQ ID NO: 102 - protein, 8 aa, synthetic construct
QQSWLGGS

SEQ ID NO: 103 - protein, 120 aa, synthetic construct [sequence omitted]

SEQ ID NO: 104 - protein, 108 aa, synthetic construct
DIQMTQSPSSLSASVGDRVTITCQASQDIGISLSWYQQKPGKAPKLLIYNANNLADGVPSRFSGSGSGTDFTLTISSLQPEDFATYYCLQHNSAPYTFGQGTKLEIKR

SEQ ID NO: 105 - protein, 5 aa, synthetic construct
DYFMN

SEQ ID NO: 106 - protein, 19 aa, synthetic construct
QMRNKNYQYGTYYAESLEG

SEQ ID NO: 107 - protein, 9 aa, synthetic construct
ESYYGFTSY

SEQ ID NO: 108 - protein, 11 aa, synthetic construct
QASQDIGISLS

SEQ ID NO: 109 - protein, 7 aa, synthetic construct
NANNLAD

SEQ ID NO: 110 - protein, 9 aa, synthetic construct
LQHNSAPYT

SEQ ID NO: 111 - protein, 8 aa, synthetic construct
GFNFNDYF

SEQ ID NO: 112 - protein, 10 aa, synthetic construct
MRNKNYQYGT

SEQ ID NO: 113 - protein, 11 aa, synthetic construct
ARESYYGFTSY

SEQ ID NO: 114 - protein, 6 aa, synthetic construct
QDIGIS

SEQ ID NO: 115 - protein, 9 aa, synthetic construct
LQHNSAPYT

SEQ ID NO: 116 - protein, 7 aa, synthetic construct
GFNFNDY

SEQ ID NO: 117 - protein, 8 aa, synthetic construct
RNKNYQYG

SEQ ID NO: 118 - protein, 9 aa, synthetic construct
ESYYGFTSY

SEQ ID NO: 119 - protein, 11 aa, synthetic construct
QASQDIGISLS

SEQ ID NO: 120 - protein, 7 aa, synthetic construct
NANNLAD

SEQ ID NO: 121 - protein, 9 aa, synthetic construct
LQHNSAPYT

SEQ ID NO: 122 - protein, 119 aa, synthetic construct [sequence omitted]

SEQ ID NO: 123 - protein, 107 aa, synthetic construct
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKR

SEQ ID NO: 124 - protein, 5 aa, synthetic construct
SFAMS

SEQ ID NO: 125 - protein, 17 aa, synthetic construct
EISSGGSYTYYPDTVTG

SEQ ID NO: 126 - protein, 10 aa, synthetic construct
GLWGYYALDY

SEQ ID NO: 127 - protein, 10 aa, synthetic construct
SASSSVSYMY

SEQ ID NO: 128 - protein, 7 aa, synthetic construct
DTSNLAS

SEQ ID NO: 129 - protein, 9 aa, synthetic construct
QQWSGYPYT

SEQ ID NO: 130 - protein, 8 aa, synthetic construct
GFTFSSFA

SEQ ID NO: 131 - protein, 8 aa, synthetic construct
ISSGGSYT

SEQ ID NO: 132 - protein, 12 aa, synthetic construct
ARGLWGYYALDY

SEQ ID NO: 133 - protein, 5 aa, synthetic construct
SSVSY

SEQ ID NO: 134 - protein, 9 aa, synthetic construct
QQWSGYPYT

SEQ ID NO: 135 - protein, 7 aa, synthetic construct
GFTFSSF

SEQ ID NO: 136 - protein, 6 aa, synthetic construct
SSGGSY

SEQ ID NO: 137 - protein, 10 aa, synthetic construct
GLWGYYALDY

SEQ ID NO: 138 - protein, 10 aa, synthetic construct
SASSSVSYMY

SEQ ID NO: 139 - protein, 7 aa, synthetic construct
DTSNLAS

SEQ ID NO: 140 - protein, 9 aa, synthetic construct
QQWSGYPYT

SEQ ID NO: 141 - protein, 119 aa, synthetic construct
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 142 - protein, 107 aa, synthetic construct
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKR

SEQ ID NO: 143 - protein, 5 aa, synthetic construct
PFAMS

SEQ ID NO: 144 - protein, 17 aa, synthetic construct
KISPGGSWTYYSDTVTG

SEQ ID NO: 145 - protein, 10 aa, synthetic construct
QLWGYYALDI

SEQ ID NO: 146 - protein, 10 aa, synthetic construct
SASISVSYMY

SEQ ID NO: 147 - protein, 7 aa, synthetic construct
DMSNLAS

SEQ ID NO: 148 - protein, 9 aa, synthetic construct
MQWSGYPYT

SEQ ID NO: 149 - protein, 8 aa, synthetic construct
GFTFSPFA

SEQ ID NO: 150 - protein, 8 aa, synthetic construct
ISPGGSWT

SEQ ID NO: 151 - protein, 12 aa, synthetic construct
ARQLWGYYALDI

SEQ ID NO: 152 - protein, 5 aa, synthetic construct
ISVSY

SEQ ID NO: 153 - protein, 9 aa, synthetic construct
MQWSGYPYT

SEQ ID NO: 154 - protein, 7 aa, synthetic construct
GFTFSPF

SEQ ID NO: 155 - protein, 6 aa, synthetic construct
SPGGSW

SEQ ID NO: 156 - protein, 10 aa, synthetic construct
QLWGYYALDI

SEQ ID NO: 157 - protein, 10 aa, synthetic construct
SASISVSYMY

SEQ ID NO: 158 - protein, 7 aa, synthetic construct
DMSNLAS

SEQ ID NO: 159 - protein, 9 aa, synthetic construct
MQWSGYPYT

SEQ ID NO: 160 - protein, 119 aa, synthetic construct
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSS

SEQ ID NO: 161 - protein, 111 aa, synthetic construct
DIVLTQSPASLALSPGERATLSCRASKSVSTSGYSYIHWYQQKPGQPPRLLIYLASNLDSGVPARFSGSGSGTDFTLNIHPLEEEDFATYYCQHSRDLPPTFGQGTKLEIK

SEQ ID NO: 162 - protein, 5 aa, synthetic construct
DYNMD

SEQ ID NO: 163 - protein, 17 aa, synthetic construct
DINPHNGGPIYNQKFTG

SEQ ID NO: 164 - protein, 10 aa, synthetic construct
GELGHWYFDV

SEQ ID NO: 165 - protein, 15 aa, synthetic construct
RASKSVSTSGYSYIH

SEQ ID NO: 166 - protein, 7 aa, synthetic construct
LASNLDS

SEQ ID NO: 167 - protein, 9 aa, synthetic construct
QHSRDLPPT

SEQ ID NO: 168 - protein, 8 aa, synthetic construct
GYTFTDYN

SEQ ID NO: 169 - protein, 8 aa, synthetic construct
INPHNGGP

SEQ ID NO: 170 - protein, 12 aa, synthetic construct
ARGELGHWYFDV

SEQ ID NO: 171 - protein, 10 aa, synthetic construct
KSVSTSGYSY

SEQ ID NO: 172 - protein, 9 aa, synthetic construct
QHSRDLPPT

SEQ ID NO: 173 - protein, 7 aa, synthetic construct
GYTFTDY

SEQ ID NO: 174 - protein, 6 aa, synthetic construct
NPHNGG

SEQ ID NO: 175 - protein, 10 aa, synthetic construct
GELGHWYFDV

SEQ ID NO: 176 - protein, 15 aa, synthetic construct
RASKSVSTSGYSYIH

SEQ ID NO: 177 - protein, 7 aa, synthetic construct
LASNLDS

SEQ ID NO: 178 - protein, 9 aa, synthetic construct
QHSRDLPPT

SEQ ID NO: 179 - protein, 119 aa, synthetic construct; identical to SEQ ID NO: 160

SEQ ID NO: 180 - protein, 111 aa, synthetic construct
DIVLTQSPASLALSPGERATLSCRASKSVSTSGYSYIHWYQQKPGQAPRLLIYLASNLDSGVPARFSGSGSGTDFTLTISSLEEEDFATYYCQHSRDLPPTFGQGTKLEIK

SEQ ID NO: 181 - protein, 5 aa, synthetic construct
DYNMD

SEQ ID NO: 182 - protein, 17 aa, synthetic construct
DINPHNGGPIYNQKFTG

SEQ ID NO: 183 - protein, 10 aa, synthetic construct
GELGHWYFDV

SEQ ID NO: 184 - protein, 15 aa, synthetic construct
RASKSVSTSGYSYIH

SEQ ID NO: 185 - protein, 7 aa, synthetic construct
LASNLDS

SEQ ID NO: 186 - protein, 9 aa, synthetic construct
QHSRDLPPT

SEQ ID NO: 187 - protein, 8 aa, synthetic construct
GYTFTDYN

SEQ ID NO: 188 - protein, 8 aa, synthetic construct
INPHNGGP

SEQ ID NO: 189 - protein, 12 aa, synthetic construct
ARGELGHWYFDV

SEQ ID NO: 190 - protein, 10 aa, synthetic construct
KSVSTSGYSY

SEQ ID NO: 191 - protein, 9 aa, synthetic construct
QHSRDLPPT

SEQ ID NO: 192 - protein, 7 aa, synthetic construct
GYTFTDY

SEQ ID NO: 193 - protein, 6 aa, synthetic construct
NPHNGG

SEQ ID NO: 194 - protein, 10 aa, synthetic construct
GELGHWYFDV

SEQ ID NO: 195 - protein, 15 aa, synthetic construct
RASKSVSTSGYSYIH

SEQ ID NO: 196 - protein, 7 aa, synthetic construct
LASNLDS

SEQ ID NO: 197 - protein, 9 aa, synthetic construct
QHSRDLPPT

SEQ ID NO: 198 - protein, 119 aa, synthetic construct; identical to SEQ ID NO: 160

SEQ ID NO: 199 - protein, 111 aa, synthetic construct; identical to SEQ ID NO: 161

SEQ ID NO: 200 - protein, 5 aa, synthetic construct
DYNMD

SEQ ID NO: 201 - protein, 17 aa, synthetic construct
DINPHNGGPIYNQKFTG

SEQ ID NO: 202 - protein, 10 aa, synthetic construct
GELGHWYFDV

SEQ ID NO: 203 - protein, 15 aa, synthetic construct
RASKSVSTSGYSYIH

SEQ ID NO: 204 - protein, 7 aa, synthetic construct
LASNLDS

SEQ ID NO: 205 - protein, 9 aa, synthetic construct
QHSRDLPPT

SEQ ID NO: 206 - protein, 8 aa, synthetic construct
GYTFTDYN

SEQ ID NO: 207 - protein, 8 aa, synthetic construct
INPHNGGP

SEQ ID NO: 208 - protein, 12 aa, synthetic construct
ARGELGHWYFDV

SEQ ID NO: 209 - protein, 10 aa, synthetic construct
KSVSTSGYSY

SEQ ID NO: 210 - protein, 9 aa, synthetic construct
QHSRDLPPT

SEQ ID NO: 211 - protein, 7 aa, synthetic construct
GYTFTDY

SEQ ID NO: 212 - protein, 6 aa, synthetic construct
NPHNGG

SEQ ID NO: 213 - protein, 10 aa, synthetic construct
GELGHWYFDV

SEQ ID NO: 214 - protein, 15 aa, synthetic construct
RASKSVSTSGYSYIH

SEQ ID NO: 215 - protein, 7 aa, synthetic construct
LASNLDS

SEQ ID NO: 216 - protein, 9 aa, synthetic construct
QHSRDLPPT

SEQ ID NO: 217 - protein, 114 aa, synthetic construct
EVQLLESGGGLVQPGGSLRLSCAASGFTFSSYGMHWVRQAPGKGLEWVAVISYDGSYKYYADSVKGRFTISRDNSKNTLYLQMNSLRAEDTAVYYCAAGVPDYWGQGTLVTVSS

SEQ ID NO: 218 - protein, 111 aa, synthetic construct
QSVLTQPPSASGTPGQRVTISCSGSSSNIGSDYDVHWYQQLPGTAPKLLIYSNEERPSGVPDRFSGSKSGTSASLAISGLRSEDEADYYCSAWDSSLSGPVFGGGTKLTVL

SEQ ID NO: 219 - protein, 5 aa, unidentified
SYGMH

SEQ ID NO: 220 - protein, 17 aa, synthetic construct
VISYDGSYKYYADSVKG

SEQ ID NO: 221 - protein, 5 aa, synthetic construct
GVPDY

SEQ ID NO: 222 - protein, 14 aa, synthetic construct
SGSSSNIGSDYDVH

SEQ ID NO: 223 - protein, 7 aa, synthetic construct
SNEERPS

SEQ ID NO: 224 - protein, 11 aa, synthetic construct
SAWDSSLSGPV

SEQ ID NO: 225 - protein, 8 aa, synthetic construct
GFTFSSYG

SEQ ID NO: 226 - protein, 8 aa, synthetic construct
ISYDGSYK

SEQ ID NO: 227 - protein, 7 aa, synthetic construct
AAGVPDY

SEQ ID NO: 228 - protein, 9 aa, synthetic construct
SSNIGSDYD

SEQ ID NO: 229 - protein, 11 aa, synthetic construct
SAWDSSLSGPV

SEQ ID NO: 230 - protein, 7 aa, synthetic construct
GFTFSSY

SEQ ID NO: 231 - protein, 6 aa, synthetic construct
SYDGSY

SEQ ID NO: 232 - protein, 5 aa, synthetic construct
GVPDY

SEQ ID NO: 233 - protein, 14 aa, synthetic construct
SGSSSNIGSDYDVH

SEQ ID NO: 234 - protein, 7 aa, synthetic construct
SNEERPS

SEQ ID NO: 235 - protein, 11 aa, synthetic construct
SAWDSSLSGPV

SEQ ID NO: 236 - protein, 117 aa, synthetic construct
QVQLVESGGGVVQPGRSLRLSCAASGFTFSHYDMHWVRQAPGKGLEWVAVISYEGSVKFYADSVKGRFTISRDNSKNTLYLQMNSLRAEDTAVYYCARTPGFHFDYWGQGTLVTVSS

SEQ ID NO: 237 - protein, 110 aa, synthetic construct
QSVLTQPPSASGTPGQRVTISCSGSSSNIGSNYVNWYQQLPGTAPKLLIYYDDLRPSGVPDRFSGSKSGTSASLAISGLRSEDEADYYCSAWDDSLSGVVFGGGTKLTVL

SEQ ID NO: 238 - protein, 5 aa, synthetic construct
HYDMH

SEQ ID NO: 239 - protein, 17 aa, synthetic construct
VISYEGSVKFYADSVKG

SEQ ID NO: 240 - protein, 8 aa, synthetic construct
TPGFHFDY

SEQ ID NO: 241 - protein, 13 aa, synthetic construct
SGSSSNIGSNYVN

SEQ ID NO: 242 - protein, 7 aa, synthetic construct
YDDLRPS

SEQ ID NO: 243 - protein, 11 aa, synthetic construct
SAWDDSLSGVV

SEQ ID NO: 244 - protein, 8 aa, synthetic construct
GFTFSHYD

SEQ ID NO: 245 - protein, 8 aa, synthetic construct
ISYEGSVK

SEQ ID NO: 246 - protein, 10 aa, synthetic construct
ARTPGFHFDY

SEQ ID NO: 247 - protein, 8 aa, synthetic construct
SSNIGSNY

SEQ ID NO: 248 - protein, 11 aa, synthetic construct
SAWDDSLSGVV

SEQ ID NO: 249 - protein, 7 aa, synthetic construct
GFTFSHY

SEQ ID NO: 250 - protein, 6 aa, synthetic construct
SYEGSV

SEQ ID NO: 251 - protein, 8 aa, synthetic construct
TPGFHFDY

SEQ ID NO: 252 - protein, 13 aa, synthetic construct
SGSSSNIGSNYVN

SEQ ID NO: 253 - protein, 7 aa, synthetic construct
YDDLRPS

SEQ ID NO: 254 - protein, 11 aa, synthetic construct
SAWDDSLSGVV

SEQ ID NO: 255 - protein, 118 aa, synthetic construct
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKIGATDPLDYWGQGTLVTVSS

SEQ ID NO: 256 - protein, 111 aa, synthetic construct
QSALTQPRSVSGSPGQSVTLSCTGTSSDVGGYNYVSWYQHYPGKAPKLMIFDVNERSSGVPDRFSGSKSGNTASLTISGLQAEDEADYYCCSYAGRYTWMFGGGTKVTVLG

SEQ ID NO: 257 - protein, 5 aa, synthetic construct
SYGMH

SEQ ID NO: 258 - protein, 17 aa, synthetic construct
VISYDGSNKYYADSVKG

SEQ ID NO: 259 - protein, 9 aa, synthetic construct
IGATDPLDY

SEQ ID NO: 260 - protein, 14 aa, synthetic construct
TGTSSDVGGYNYVS

SEQ ID NO: 261 - protein, 7 aa, synthetic construct
DVNERSS

SEQ ID NO: 262 - protein, 10 aa, synthetic construct
CSYAGRYTWM

SEQ ID NO: 263 - protein, 8 aa, synthetic construct
GFTFSSYG

SEQ ID NO: 264 - protein, 8 aa, synthetic construct
ISYDGSNK

SEQ ID NO: 265 - protein, 11 aa, synthetic construct
AKIGATDPLDY

SEQ ID NO: 266 - protein, 9 aa, synthetic construct
SSDVGGYNY

SEQ ID NO: 267 - protein, 10 aa, synthetic construct
CSYAGRYTWM

SEQ ID NO: 268 - protein, 7 aa, synthetic construct
GFTFSSY

SEQ ID NO: 269 - protein, 6 aa, synthetic construct
SYDGSN

SEQ ID NO: 270 - protein, 9 aa, synthetic construct
IGATDPLDY

SEQ ID NO: 271 - protein, 14 aa, synthetic construct
TGTSSDVGGYNYVS

SEQ ID NO: 272 - protein, 7 aa, synthetic construct
DVNERSS

SEQ ID NO: 273 - protein, 10 aa, synthetic construct
CSYAGRYTWM

SEQ ID NO: 274 - protein, 120 aa, synthetic construct
EVQLVESGGGLVQPGGSLRLSCAASGFTFSSFGMHWVRQAPGKGLEWVAYITPGSNTIYYADTVKGRFTISRDNAKNSLYLQMNSLRAEDTAVYYCAREYYSYDEGFAYWGQGTLVTVSS

SEQ ID NO: 275 - protein, 108 aa, synthetic construct
EIVLTQSPATLSLSPGERATLSCSVSDLIPSQYLHWYQQKPGQAPRLLIYRTSNLASGIPARFSGSGSGTDFTLTISSLEPEDFAVYYCQQGVDVPFTFGQGTKLEIK

SEQ ID NO: 276 - protein, 5 aa, synthetic construct
SFGMH

SEQ ID NO: 277 - protein, 17 aa, synthetic construct
YITPGSNTIYYADTVKG

SEQ ID NO: 278 - protein, 11 aa, synthetic construct
EYYSYDEGFAY

SEQ ID NO: 279 - protein, 12 aa, synthetic construct
SVSDLIPSQYLH

SEQ ID NO: 280 - protein, 7 aa, synthetic construct
RTSNLAS

SEQ ID NO: 281 - protein, 9 aa, synthetic construct
QQGVDVPFT

SEQ ID NO: 282 - protein, 8 aa, synthetic construct
GFTFSSFG

SEQ ID NO: 283 - protein, 8 aa, synthetic construct
ITPGSNTI

SEQ ID NO: 284 - protein, 13 aa, synthetic construct
AREYYSYDEGFAY

SEQ ID NO: 285 - protein, 7 aa, synthetic construct
DLIPSQY

SEQ ID NO: 286 - protein, 9 aa, synthetic construct
QQGVDVPFT

SEQ ID NO: 287 - protein, 7 aa, synthetic construct
GFTFSSF

SEQ ID NO: 288 - protein, 6 aa, synthetic construct
TPGSNT

SEQ ID NO: 289 - protein, 11 aa, synthetic construct
EYYSYDEGFAY

SEQ ID NO: 290 - protein, 12 aa, synthetic construct
SVSDLIPSQYLH

SEQ ID NO: 291 - protein, 7 aa, synthetic construct
RTSNLAS

SEQ ID NO: 292 - protein, 9 aa, synthetic construct
QQGVDVPFT

SEQ ID NO: 293 - protein, 117 aa, synthetic construct
EVQLVQSGAEVKKPGASVKVSCKASGYTFTHYNMDWVRQAPGQGLEWMGTIYPGNADTSYNQKFKGRVTMTRDTSTSTVYMELSSLRSEDTAVYYCARGDLYALDYWGQGTLVTVSS

SEQ ID NO: 294 - protein, 107 aa, synthetic construct
DIQMTQSPSSLSASVGDRVTITCKASQSVYRDVAWYQQKPGKAPKLLIYSASNRYTGVPSRFSGSGSGTDFTFTISSLQPEDIATYYCQHDYSSPYTFGQGTKLEIK

SEQ ID NO: 295 - protein, 5 aa, synthetic construct
HYNMD

SEQ ID NO: 296 - protein, 17 aa, synthetic construct
TIYPGNADTSYNQKFKG

SEQ ID NO: 297 - protein, 8 aa, synthetic construct
GDLYALDY

SEQ ID NO: 298 - protein, 11 aa, synthetic construct
KASQSVYRDVA

SEQ ID NO: 299 - protein, 7 aa, synthetic construct
SASNRYT

SEQ ID NO: 300 - protein, 9 aa, synthetic construct
QHDYSSPYT

SEQ ID NO: 301 - protein, 8 aa, synthetic construct
GYTFTHYN

SEQ ID NO: 302 - protein, 8 aa, synthetic construct
IYPGNADT

SEQ ID NO: 303 - protein, 10 aa, synthetic construct
ARGDLYALDY

SEQ ID NO: 304 - protein, 6 aa, synthetic construct
QSVYRD

SEQ ID NO: 305 - protein, 9 aa, synthetic construct
QHDYSSPYT

SEQ ID NO: 306 - protein, 7 aa, synthetic construct
GYTFTHY

SEQ ID NO: 307 - protein, 6 aa, synthetic construct
YPGNAD

SEQ ID NO: 308 - protein, 8 aa, synthetic construct
GDLYALDY

SEQ ID NO: 309 - protein, 11 aa, synthetic construct
KASQSVYRDVA

SEQ ID NO: 310 - protein, 7 aa, synthetic construct
SASNRYT

SEQ ID NO: 311 - protein, 9 aa, synthetic construct
QHDYSSPYT

SEQ ID NO: 312 - protein, 118 aa, synthetic construct [sequence omitted]

SEQ ID NO: 313 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 314 - protein, 5 aa, synthetic construct
DSWIH

SEQ ID NO: 315 - protein, 17 aa, synthetic construct
WISPYGGSTYYADSVKG

SEQ ID NO: 316 - protein, 9 aa, synthetic construct
RHWPGGFDY

SEQ ID NO: 317 - protein, 11 aa, synthetic construct
RASQDVSTAVA

SEQ ID NO: 318 - protein, 7 aa, synthetic construct
SASFLYS

SEQ ID NO: 319 - protein, 9 aa, synthetic construct
QQYLYHPAT

SEQ ID NO: 320 - protein, 8 aa, synthetic construct
GFTFSDSW

SEQ ID NO: 321 - protein, 8 aa, synthetic construct
ISPYGGST

SEQ ID NO: 322 - protein, 11 aa, synthetic construct
ARRHWPGGFDY

SEQ ID NO: 323 - protein, 6 aa, synthetic construct
QDVSTA

SEQ ID NO: 324 - protein, 9 aa, synthetic construct
QQYLYHPAT

SEQ ID NO: 325 - protein, 7 aa, synthetic construct
GFTFSDS

SEQ ID NO: 326 - protein, 6 aa, synthetic construct
SPYGGS

SEQ ID NO: 327 - protein, 9 aa, synthetic construct
RHWPGGFDY

SEQ ID NO: 328 - protein, 11 aa, synthetic construct
RASQDVSTAVA

SEQ ID NO: 329 - protein, 7 aa, synthetic construct
SASFLYS

SEQ ID NO: 330 - protein, 9 aa, synthetic construct
QQYLYHPAT

SEQ ID NO: 331 - protein, 120 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 332 - protein, 113 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQP

SEQ ID NO: 333 - protein, 5 aa, synthetic construct
SYIMM

SEQ ID NO: 334 - protein, 17 aa, synthetic construct
SIYPSGGITFYADTVKG

SEQ ID NO: 335 - protein, 11 aa, synthetic construct
IKLGTVTTVDY

SEQ ID NO: 336 - protein, 14 aa, synthetic construct
TGTSSDVGGYNYVS

SEQ ID NO: 337 - protein, 7 aa, synthetic construct
DVSNRPS

SEQ ID NO: 338 - protein, 10 aa, synthetic construct
SSYTSSSTRV

SEQ ID NO: 339 - protein, 8 aa, synthetic construct
GFTFSSYI

SEQ ID NO: 340 - protein, 8 aa, synthetic construct
IYPSGGIT

SEQ ID NO: 341 - protein, 13 aa, synthetic construct
ARIKLGTVTTVDY

SEQ ID NO: 342 - protein, 9 aa, synthetic construct
SSDVGGYNY

SEQ ID NO: 343 - protein, 10 aa, synthetic construct
SSYTSSSTRV

SEQ ID NO: 344 - protein, 7 aa, synthetic construct
GFTFSSY

SEQ ID NO: 345 - protein, 6 aa, synthetic construct
YPSGGI

SEQ ID NO: 346 - protein, 11 aa, synthetic construct
IKLGTVTTVDY

SEQ ID NO: 347 - protein, 14 aa, synthetic construct
TGTSSDVGGYNYVS

SEQ ID NO: 348 - protein, 7 aa, synthetic construct
DVSNRPS

SEQ ID NO: 349 - protein, 10 aa, synthetic construct
SSYTSSSTRV

SEQ ID NO: 350 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 351 - protein, 111 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTV

SEQ ID NO: 352 - protein, 5 aa, synthetic construct
RYWMS

SEQ ID NO: 353 - protein, 17 aa, synthetic construct
NIKQDGSEKYYVDSVKG

SEQ ID NO: 354 - protein, 12 aa, synthetic construct
EGGWFGELAFDY

SEQ ID NO: 355 - protein, 12 aa, synthetic construct
RASQRVSSSYLA

SEQ ID NO: 356 - protein, 7 aa, synthetic construct
DASSRAT

SEQ ID NO: 357 - protein, 9 aa, synthetic construct
QQYGSLPWT

SEQ ID NO: 358 - protein, 8 aa, synthetic construct
GFTFSRYW

SEQ ID NO: 359 - protein, 8 aa, synthetic construct
IKQDGSEK

SEQ ID NO: 360 - protein, 14 aa, synthetic construct
AREGGWFGELAFDY

SEQ ID NO: 361 - protein, 7 aa, synthetic construct
QRVSSSY

SEQ ID NO: 362 - protein, 9 aa, synthetic construct
QQYGSLPWT

SEQ ID NO: 363 - protein, 7 aa, synthetic construct
GFTFSRY

SEQ ID NO: 364 - protein, 6 aa, synthetic construct
KQDGSE

SEQ ID NO: 365 - protein, 12 aa, synthetic construct
EGGWFGELAFDY

SEQ ID NO: 366 - protein, 12 aa, synthetic construct
RASQRVSSSYLA

SEQ ID NO: 367 - protein, 7 aa, synthetic construct
DASSRAT

SEQ ID NO: 368 - protein, 9 aa, synthetic construct
QQYGSLPWT

SEQ ID NO: 369 - protein, 636 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 370 - protein, 636 aa, synthetic construct
DKTHTCPPCPAPEAARAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPEAARAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 371 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 372 - protein, 330 aa, synthetic construct [sequence omitted]

SEQ ID NO: 373 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 374 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 375 - protein, 107 aa, synthetic construct [sequence omitted]

SEQ ID NO: 376 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 377 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 378 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 379 - protein, 106 aa, synthetic construct
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 380 - protein, 106 aa, synthetic construct
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS